(19) United States
(12) Patent Application Publication
(10) Pub. No.: US 2006/0275847 A1
(43) Pub. Date: Dec. 7, 2006

(54) Method for assessing biofilms

(76) Inventors: Ian David Goodyer, Whitechurch (GB); Rudi Labarbe, Cardiff (GB); Dietrich Ruehlmann, Gaithersburg, MD (US); Simon Stubbs, Cardiff (GB)

Correspondence Address: GE HEALTHCARE BIO-SCIENCES CORP.;PATENT DEPARTMENT, 800 CENTENNIAL AVENUE, PISCATAWAY, NJ, 08855, US

(21) Appl. No.: 10/514,138

(22) PCT Filed: Apr. 28, 2003
(86) PCT No.: PCT/GB03/01816

§ 371 Date: May 9, 2005

(30) Foreign Application Priority Data
May 14, 2002 (GB) 0211068.2

(51) Int. Cl. G01N 33/554; C12Q 1/34; C12Q 1/42; C12Q 1/26; C12Q 1/04

(52) U.S. Cl. 435/7.32; 435/18; 435/21; 435/25; 435/34

(57) ABSTRACT

An automated method for measuring the development of a biofilm, containing one or more fluorescent moieties, on a plurality of surfaces using a confocal imaging system including: a) a radiation source system for forming a beam of electromagnetic radiation including one or more wavelengths; b) an optical system for directing and focusing said beam onto one or more planes of the object; c) a detection system for detecting electromagnetic radiation emitted from the object and producing image data; and d) a scanning system for scanning the object in a plurality of planes with the electromagnetic radiation, the method comprising the steps of: i) growing said biofilm on said plurality of surfaces; ii) detecting the presence of said one or more fluorescent moieties within the biofilm by scanning the biofilm with electromagnetic radiation in a plurality of planes and collecting fluorescent emissions to produce a plurality of images; and iii) analysing said images by means of a data processing system under the control of computer software to determine the structure of the biofilm.

### TECHNICAL FIELD

The present invention relates to a method for automatically measuring the development of a microbial biofilm using a confocal imaging system and to methods for determining the effect of test chemicals on microbial gene expression and biofilm development.

### BACKGROUND TO INVENTION

Microbial biofilms consist of homogeneous or heterogeneous microbial populations adhering to surfaces or interfaces, usually embedded in an extracellular matrix of polysaccharides (Costerton et al., 1995, Annual Review of Microbiology, 41, 435-464). These biofilms can form rapidly on almost any wet surface and represent the normal mode of colonisation of microbes in the environment (Wood et al., 2000, Journal of Dental Research, 79, 21-27). Although bacteria are frequently associated with biofilm development, many microbes including fungi and algae also form biofilms.

Microbial biofilms cause widespread problems in industry, fouling machinery, clogging piping and adhering to the hulls of marine equipment and shipping. Biofilms are also a significant problem in medicine, being implicated in a large number of human infections such as dental caries, periodontitis and cystic fibrosis pneumonia (Costerton et al., 1999, Science, 284, 1318-1322). Infections arising from contamination of medical equipment are often due to bacteria present as biofilms (Gornan et al., 1994, Epidemiological Journal, 112, 551-559).

Bacteria in biofilms often display markedly different phenotypes compared to their free-swimming planktonic counterparts which can give rise to serious problems in industry and medicine. Of greatest significance is their increased tolerance to antibiotic treatment. Soukos et al. (Pharmaceutical Research, 2000, 17, 405-409) report that bacteria within biofilms can be 1500-fold less sensitive to antibiotic treatment compared to planktonic cells of the same species.

Recent research has shown that molecules, termed ‘quorum-sensing signals’, are constantly secreted by microbes and activate genes involved in the production of the extracellular matrix and biofilm formation (Chicurel, 2000, Nature, 408,284-286). Efforts have now been directed into producing molecular mimics of these naturally occurring signals to bind to the microbial receptor sites and thus control biofilm formation (Costerton & Stewart, 2001, Scientific American, July, 61-65).

The control of microbial biofilms thus poses significant challenges for many industries, including the food, health, consumer products, engineering and pharmaceutical industries. In the last decade considerable efforts have been marshalled to address this issue but attempts to discover and develop novel antimicrobial agents effective against biofilms have been hampered by a lack of a suitable screening assay. While planktonic microbes are readily amenable to high throughput screening technologies, the growth and assessment of biofilm sensitivity to inhibitors, quorum-sensing signal mimics or agonists is laborious, time consuming and beset with technical difficulties.

Any assay or method to determine the effect that an agent has upon the growth and development of a biofilm, by necessity, involves an assessment of its development and architecture. Electron microscopy has traditionally been the method of choice for studying biofilm composition and structure because of its high resolution (Listgarten, 1976, Journal of Periodontology, 47, 1-18). However, this technique is time consuming, can cause structural distortions through the preparation process and is not amenable to high throughput screening.

Many of the above problems associated with electron microscopy have been addressed by the advent of laser-scanning confocal microscopy (LSCM) which enables biofilm structure to be studied in its natural state without any requirement for dehydration, fixation or staining. The optical sectioning properties of LSCM enables very thin optical sections (approximately 0.3 μm) to be taken at increasing depths throughout the biofilm, free from out-of-focus blurring. Intrinsically fluorescent molecules (e.g. green fluorescent proteins) or fluorescently labelled probes are excited and the resulting fluorescence detected by photomultiplier tubes to produce a digital image. The digitised data can then be reassembled to provide three-dimensional (3D) information on the structure. Confocal microscopy has now been used to investigate biofilm structure (Wood et al., 2000, Journal of Dental Research 79, 21-27), physiology and biochemistry (Palmer & Stemberg, 1999, Current Opinion in Biotechnology 10, 263-268). However, such studies are time consuming and have been highly specific in nature, concentrating on only one or two specialised biofilm structures.

While LSCM provides an excellent tool for investigating biofilm morphology, structure and composition, it is not an obvious choice as a platform for high throughput screening because the imaging and data capture process is very slow. Kuehn et al. (Applied and Environmental Microbiology, 1998, 64, 4115-4127) report an ‘automated’ LSCM for analysing biofilms, the hub of the invention being a computer program for semi-automated image analysis. This system is based upon a point scan detection method and involves ‘off-line’ semi-automated image analysis requiring user input/intervention. These data capture and analytical methods, together with the use of ‘glass flow cells’ for biofilm growth, severely limit the applicability of this approach to automation and high throughput screening. Other screening methods for assessing the effects of test agents on biofilms are disclosed in U.S. Pat. No. 6,326,190, wherein a variety of methods such as colony counting and vital staining are used to determine biofilm growth. Of particular note, however, are the problems highlighted in automating any assay using confocal microscopy.

The present invention addresses the above mentioned problems associated with providing a LSCM-based method for analysing biofilm development which is amenable to automation. Furthermore, the method of the present invention can provide a platform for high throughput screening for novel modulators of biofilm growth and development. In contrast to the autofocus methods based upon image content analysis described in the literature (e.g. WO 96/01438), the present invention utilises a position sensing analysis.

WO 99/47963 discloses a ‘Confocal Microscopy Imaging System’ for identifying pharmacological agents useful for the diagnosis and treatment of disease by performing a variety of assays on cell extracts, cells or tissues of higher organisms using the line-scan confocal imaging system and associated data processing routines. The imaging system can be used to perform multi-parameter fluorescence imaging on single cells and populations in a manner that is sufficiently rapid for compound screening. The system is capable of determining the presence of fluorophores with high resolution. However, nowhere within this application is there any disclosure of the use of the system to characterise microbiological populations, to create 3D images thereof, or to analyse biofilm development. Furthermore, the image analysis algorithms described in WO 99/47963 are only suitable for analysing data from a single plane and not a plurality of planes.

The Applicants have found that confocal imaging systems, such as that described in WO 99/47963, when used in combination with the image analytical methods of the present invention, can be used to characterise biofilms and to act as a platform for high throughput screening for compounds which modulate biofilm development. The IN Cell Analyzer and its use in high throughput screening applications is reported by Goodyer et al.,2001, Society for Biomolecular Screening, 7^{th} Annual Conference and Exhibition, Baltimore, USA Screening and signalling events in live cells using novel GFP redistribution assays.

### SUMMARY OF THE INVENTION

According to a first aspect of the present invention, there is provided an automated method for measuring the development of a biofilm on a plurality of surfaces using a confocal imaging system, the confocal imaging system comprising:
a) means for forming a beam of electromagnetic radiation comprising one or more wavelengths;
b) means for directing and focusing said beam onto one or more planes of a biofilm;
c) a detection device for detecting electromagnetic radiation emitted from the biofilm; and
d) a scanning device for scanning the biofilm in a plurality of planes with the electromagnetic radiation,
   the method comprising the steps of:
i) growing the biofilm on the plurality of surfaces;
ii) detecting the presence of one or more fluorescent moieties within the biofilm by scanning the biofilm with electromagnetic radiation in a plurality of planes to produce a plurality of images; and
iii) analysing the images by means of a data processing system under the control of computer software to determine the structure of the biofilm.

Biofilms are dynamic structures which are responsive to their environment (Watnick & Kolter, 2000, Journal of Bacteriology, 182, 2675-2679). In the context of the present invention, the word ‘development’, when used in relation to a biofilm, is to be construed to describe the growth, stasis or the deterioration of the biofilm.

In a preferred embodiment, as a means for increasing speed of data acquisition:
a) the beam forming means produces an elongated beam of electromagnetic radiation comprising one or more wavelengths and extending transverse to an optical axis along which the radiation propagates;
b) the directing and focusing means focuses the elongated beam onto a first elongated region in a first plane where the biofilm is located and direct electromagnetic radiation emitted from the biofilm onto one or more second elongated regions, wherein each second elongated region is on a different second plane conjugate to the first plane;
c) in at least one of the second conjugate planes, or in a third plane conjugate to at least one of the second conjugate planes, the detection device comprises a rectangular array of detection elements on which the electromagnetic radiation emitted from the object is coincident; and
d) the scanning device scans the biofilm by moving the elongated beam relative to the biofilm or by moving the biofilm relative to the elongated beam such that the emitted electromagnetic radiation is delivered to the rectangular array of detection elements and is converted by the detection device into a plurality of electrical signals representative of the emitted electromagnetic radiation synchronously with the scanning.

Preferably, the method additionally comprises the step of restoring each image prior to carrying out the image analysis. Image restoration refers to the problem of recovering an image from its blurred and noisy observation for the purpose of improving its quality or obtaining information that is not readily available from the observed image. Factors influencing the spatial resolution are mainly scattering of the emitted photons and aberrations and distortions introduced by the imaging system. If the object to be imaged is small compared to the source-to-collimator distance, this degradation phenomenon may be considered to be approximately shift-invariant and, neglecting noise, can be modelled by a convolution process between the undistorted image and the transfer function of the imaging system. Many types of image restoration have been reported in the literature. For example, Wiener filter, wavelet-based regularisation, supervised deconvolution methods, iterative blind deconvolution methods, etc. For a comparison between the different methods see, for example, Lixin Shen, 2002, Journal of Electronic Inaging, 11, 5-10 or Mignotte et al., 2002, Journal of Electronic Imaging (2002), 11, 11-24.

Suitably, the beam of electromagnetic radiation produced comprises one or more wavelengths in the range of 350 to 700 nm. Preferred ranges in wavelength include 354 to 374 nm, 403 to 423 nm, 478 to 498 mn, 560 to 580 nm, 637 to 657 nm and 680 to 700 nm. Preferred wavelengths include 364 nm, 413 nm, 488 nm, 570 nm, 647 nm and 690 nm.

Suitably, the fluorescent moiety is an inherent characteristic of the microbe within the biofilm.

Preferably, the fluorescent moiety is the product of a gene that is expressed by the microbe within the biofilm. For example, the microbe may have been genetically transformed to express the gene in a constitutive or an inducible manner. More preferably, the gene may contain codons that have been altered to optimise expression of the fluorescent moiety in the microbe.

Preferably, the gene encodes a fluorescent protein. Fluorescent proteins and fluorescent protein derivatives of chromoproteins have been isolated from a wide variety of organisms, including Aequoria victoria, Anemonia species such as A. majano and A. sulcata, Renilla species, Ptilosarcus species, Discosoma species, Claularia species, Dendronephthyla species, Ricordia species, Scolymia species, Zoanthus species, Montastraea species, Heteractis species, Conylactis species and Goniopara species.

The use of Green Fluorescent Protein (GFP) derived from Aequorea victoria has revolutionised research into many cellular and molecular-biological processes. However, as the fluorescence characteristics of wild type (native) GFP (wtGFP) are not ideally suited for use as a cellular reporter, significant effort has been expended to produce variant mutated forms of GFP with properties more suitable for use as an intracellular reporter (Heim et al., 1994, Proceedings of the National Academy of Sciences (USA), 91, 12501;. Ehrig et al., 1995, FEBS Letters, 367,163-6; WO96/27675; Crameri, A. et al., 1996, Nature Biotechnology, 14, 315-9; U.S. Pat. No. 6,172,188; Cormack, B. P. et al., 1996, Gene, 173, 33-38; U.S. Pat. No. 6,194,548; U.S. Pat. No. 6,077,707 and GB Patent Application Number 0109858.1 (‘Amersham Pharmacia Biotech UK Ltd.’). Preferred embodiments disclosed in GB Patent Application No 0109858.1 comprise GFP derivatives selected from the group consisting of: F64L-V163A-E222G-GFP, F64L-S 175G-E222G-GFP, F64L-S65T-S 175G-GFP and F64L-S65T-V163A-GFP.

Preferably, the fluorescent protein is a modified GFP having one or more mutations selected from the group consisting of Y66H, Y66W, Y66F, S65T, S65A, V68L, Q69K, Q69M, S72A, T2031, E222G, V163A, I167T, S175G, F99S, M153T, V163A, F64L, Y145F, N149K, T203Y, T203Y, T203H, S202F and L236R. Most preferably, the fluorescent protein is a modified GFP having three mutations selected from the group consisting of F64L-V163A-E222G, F64L-S175G-E222G, F64L-S65T-S175G and F64L-S65T-V163.

Preferably, the fluorescent moiety is a biosensor capable of monitoring environmental change or enzyme activity within the biofilm. For example, the moiety may be a fluorescent biosensing protein that is responsive to pH changes (e.g., Llopsis et al., 1998, Proceedings of the National Academy of Sciences (USA) 95, 6803-6808) or may be sensitive to specific ion concentrations, such as calcium ions, within the biofilm.

Optionally, the fluorescent moiety is produced by the action of an enzyme on a compound. Preferably, the enzyme is selected from the group consisting of β-galactosidase, nitroreductase, alkaline phosphatase and β-lactamase. The enzyme may be inherently expressed by the micro-organism or the micro-organism may have been genetically transformed to express the enzyme in an inducible or constitutive manner.

In a preferred embodiment, the method additionally comprises adding a fluorescent compound to the biofilm before carrying out the detection step.

Preferably, the fluorescent compound is selected from the group consisting of Hoechst 33342, Cy2, Cy3, Cy5, CytoCyS, CypHer, coumarin, FITC, DAPI, Alexa 633 DRAQ5, Alexa 488, acridone, quinacrodone, fluorescently labelled protein, fluorescently labelled lectin and fluorescently labelled antibody. By ‘acridone’ and ‘quinacridone’ is meant those fluorescent compounds disclosed in WO 02/099424 and 02/099432, respectively. Optionally, unbound fluorescent compound can be removed from each container prior to carrying out the detection step.

Preferably, the fluorescent compound can be used to monitor environmental changes present throughout the biofilm. Thus, for example, the redox potential and pH of the microbial biofllm are subject to local environmental changes which can be measured using the present invention. pH sensitive dyes, such as the CyDye ‘CypHer’ (cf. Amersham Biosciences, reference PA15405), can be used to determine changes in pH within the biofilm. For example, in comparison with Cy5, CypHer has 95% fluorescence at pH 5.0 but only 5% fluorescence at pH 7.4, thereby allowing quantitative measurement of variations in local pH within the biofilm.

Optionally, the fluorescent compound can be used to measure the concentration of a chemical within or surrounding the biofilm, such as the concentration of oxygen or calcium, or of a specific protein or nucleic acid.

Preferably, the surfaces are in the form of a container. More preferably, the container is a microtitre plate. The microtitre plate may comprise 24, 96, 384 or higher densities of wells e.g. 864 or 1536 wells. Preferably the microtitre plates have a transparent base as imaging of the biofilm is conducted through the base (Gilbert et al., 2001, Journal of Applied Microbiology, 91, 248-254) Suitably, the method is capable of determining the size of microbial clusters within the biofilm by means of optically and confocally sectioning the biofilm followed by 3D volume analysis.

Suitably, the method is capable of determining the 3D structure of the biofilm by means of optically and confocally sectioning the biofilm followed by 3D volume analysis.

Suitably, the method is capable of determining the distribution of distances between microbial clusters within the biofilm by means of optically and confocally sectioning the biofilm followed by 3D volume analysis.

Suitably, the method is capable of determining the orientation of microbial clusters within the structure of the biofilm. The orientation of the clusters may be influenced by external forces such as fluid flow, gravity, electric field, etc.

Suitably, the method is capable of determining the presence of and the size of any channel within the structure of the biofilm. Such channels provide access routes for nutrients into, and exit routes for noxious waste from, the biofilm.

Thus, for example, channels can be readily identified in the biofilm structure, as can be seen from FIGS. 16A, C and D, where the black areas represent channels. Preferably, the method is capable of determining the connectivity of the channel with any other channel within the structure of the biofilm, thereby defining a channel network. More preferably, the method is capable of determining the fractal dimension of the channel network.

A biofilm can be thought of as a porous medium, in which different molecules can diffuse in the network of channels between the clusters of micro-organism. It is important to be able to relate the geometrical structure of the network with the diffision properties through the network as this will give an indication of the ease with which nutrients, oxygen, antibiotic agents or other molecules can move inside the biofilm. A porous medium is a material randomly multi-connected in which channels are randomly blocked. The fraction of free space occupied by these channels is called the porosity. The Darcy permeability of the channel network is the equivalent of the conductivity of a network of resistors; it describes how easily a liquid can flow through the network. Above a value of the porosity called the critical porosity C_{cr}, there is a continuous path through the network and the molecules can diffuse from one end of the network to the other. If the distribution of the channels is random and the long distance correlations are negligible, the permeability follows a universal power law close to C_{cr}, that does not depend on the microscopic details, nature of the connections, etc. The flow of liquid through the network is linked to the permeability of the network, therefore ultimately at the global geometry of the network. In many cases, the geometry of the network can be described as a fractal and a fractal dimension can be defined for the network. At the critical value, the average mass of the channel included in a sphere of radius R is proportional to R^{D}, with D<3. The network can thus be thought of as a porous structure that fills the 3 dimensional space less efficiently than a usual three dimensional solid and it could be said the network has a fractal dimension, which is less than the usual Euclidian dimension. In summary, determining the fractal dimension gives a description of the geometry of the network and therefore gives an indicator on the porosity of the biofilm. For more details see, for example, Gouyet, Physique et Structure Fractales, Publisher Mason, 1992, Chapter 3.

In another embodiment, the biofilm comprises optically distinguishable microbial populations. These populations may, for example, comprise the same or different microbial species which may be differentiated on the basis of different labelling patterns on treatment with a fluorescent label. Preferably, the method is capable of determining the spatial distribution of said populations.

In a further embodiment, the biofilm comprises genetically distinguishable microbial populations. These populations may comprise different strains or species; for example, the population may consist of a wild type and a genetically engineered bacterial strain that constitutively expresses GFP, or consist of two or more strains or species that express optically distinct fluorescent reporter genes in an inducible or constitutive manner. Preferably, the method is capable of determining the spatial distribution of gene expression and the populations.

According to a second aspect of the present invention, there is provided a method for screening a test agent whose effect upon the development of a biofilm is to be determined, the method comprising the steps of:
i) performing the method as hereinbefore described in the presence of the test agent; and
ii) comparing the development of the biofilm in the presence of the test agent with a known value for the development of the biofilm in the absence of the test agent,
   wherein a difference between the development of the biofilm in the presence of the test agent and the known value in the absence of the test agent is indicative of the effect of the test agent upon the development of the biofilm.

Preferably the known value is stored upon an optical or electronic database. Optionally, the value may be normalised (for example, to represent 100% development of the biofilm) and compared to the normalised development of the biofilm in the presence of the test agent. In this way, only test agents affecting biofilm development by a certain minimum amount will be selected for further evaluation.

In a third aspect of the present invention, there is provided a method for screening a test agent whose effect upon the development of a biofilm is to be determined, the method comprising the steps of:
i) growing the biofilm in the presence and absence of the test agent; and
ii) measuring the development of the biofilm according to the method as hereinbefore described,
   wherein a difference in the development the biofilm in the presence and absence of the agent is indicative of the effect the test agent has upon the development of the biofilm.

Thus, for example, antibiotics such as tetracycline, ampicillin and chloramphenicol can be shown to differentially inhibit biofilm development (see FIGS. 17 and 18).

Preferably, the difference in the development of the biofilm in the absence and in the presence of the test agent is normalised, stored optically or electronically and compared with a value of a reference compound. Thus, for example, the difference in development may be stored as a percentage inhibition (or percentage stimulation) on an electronic database and this value compared to the corresponding value for a standard inhibitor of the biofilm in question. In this way, only test agents meeting a certain predetermined threshold (e.g. as being as effective as or more effective than the reference compound) may be selected as being of interest for further testing.

Suitably, the test agent affects gene expression within the biofilm. Preferably, the test agent affects gene expression of specific microbial populations within the biofilm.

Suitably, the test agent inhibits biofilm development.

Suitably, the test agent promotes the development of the biofilm. Such compounds would be of particular interest for industries such as the water treatment and sewage industries.

Preferably, the test agent is a physical agent selected from the group consisting of electromagnetic radiation, ionising radiation, electric field, sound energy and abrasion. Suitable forms of electromagnetic radiation would include ultra violet radiation, while suitable forms of ionising radiation would include α-, β- and γ-radiation. Abrasion would involve physically rubbing or scraping a physical entity, in the form of a mechanical object such as a brush or particulate material, against the surface of the biofilm.

Preferably, the test agent is a fluorescent compound or is a fluorescently labelled compound thereby facilitating measurement of its distribution throughout the biofilm. More preferably, the test agent is selected from the group consisting of organic compound, inorganic compound, peptide, protein, carbohydrate, lipid, nucleic acid, polynucleotide and protein nucleic acid.

According to a fourth aspect of the present invention, there is provided the use of a confocal imaging system as herein described to measure the development of a biofilm.

According to a fifth aspect of the invention there is provided a method of analysing the three dimensional structure of an object scanned using a fluorescence imaging system including:
a) a radiation source system for forming a beam of electromagnetic radiation comprising one or more wavelengths;
b) an optical system for directing and focusing said beam onto one or more planes of the object;
c) a detection system for detecting electromagnetic radiation emitted from the object and producing image data; and
d) a scanning system for scanning the object in a plurality of planes with the electromagnetic radiation,

the method comprising processing said image data to determine data relating to a three dimensional structure of the object, the method including an automated image data thresholding step, said thresholding step comprising:
i) analysing intensity values in the image data;
ii) calculating a threshold value for the image data; and
iii) processing the image data using said threshold value to generate thresholded image data.

The automated determination of a threshold for each of a plurality of different images enables a high throughput image analysis system for analysing three dimensional structures in image data produced by a fluorescence imaging system. This aspect in particular but not exclusively applies to biofilms. By automated thresholding on a per-image basis, or for each of a set of images taken of a biofilm in different planes, it is possible to overcome problems including: a variation in an average image intensity for different samples of the biofilm with for example, different experimental parameters such as a different dye concentration, a different laser power or a different camera exposure time; a variation in an image intensity for images of different planes of the biofilm sample due to a difference in depth of the planes within the sample; and a regional intensity variation of an image due to for example, a non uniform illumination of a sample or a non uniform variation of the sample thickness or density.

According to sixth and seventh aspects of the present invention, there is provided computer software and a data carrier storing such computer software, respectively, for carrying out the method of the invention as described herein.

### BRIEF DESCRIPTION OF THE DRAWINGS

The invention is further described with reference to the following drawings in which:

FIG. 1 is a schematic view of a line-scan confocal microscope used to image biofilms according to the present invention.

FIGS. 2(a) and 2(b) are, respectively, a top view and a side view of the ray path of a multicolour embodiment of the present invention, without a scanning mirror.

FIG. 2(c) is a top view of the ray path of a single beam autofocus system.

FIGS. 3(a) and 3(b) are, respectively, a top view and a side view of the ray path of the multicolour embodiment of the present invention with the scanning mirror.

FIG. 3(c) is a top view of the ray path of the single beam autofocus system.

FIG. 4 is a side view of the two beam autofocus system.

FIGS. 5(a) to 5(c) illustrate the rectangular CCD camera and readout register.

FIG. 6 illustrates schematically data processing components of an imaging data processing system.

FIG. 7 represents a flow diagram of an image analysis procedure according to an embodiment of the invention.

FIG. 8 represents a flow chart of an image data binarising step of the analysis procedure

FIGS. 9(a) and 10(a) each illustrate an image of a plane of a biofilm scanned in a different colour channel.

FIGS. 9(b) and 10(b) each illustrate an intensity histogram for the image of the plane of the biofilm of FIGS. 9(a) and 10(a).

FIGS. 9(c) and 10(c) each illustrate a binarised image of the plane of the biofilm of FIGS. 9(a), 9(b), 10(a) and 10(b).

FIG. 11 illustrates an alternative image data thresholding step of the analysis procedure.

FIG. 12 depicts a flow chart of a sub-routine of the procedure shown in FIG. 7.

FIG. 13 represents a flow chart of the 3D object analysis sub-routine.

FIG. 14 illustrates a flow chart of the 3D object cross correlation analysis sub-process.

FIG. 15 is a micrograph showing an E. coli biofilm stained with the fluorescent dye Hoechst 33342.

FIGS. 16A-D represent a 3D analysis of a GFP expressing biofilm;

FIGS. 16A, C and D representing 4, 9 & 14 μm slices and FIG. 16D representing a 3D image. FIG. 16E illustrates the variation in the volume of green and non-green bacteria as a function of the position the cells in the Z-plane.

FIG. 17 depicts the differential inhibition of adhered cultures with selected antibiotics. Filled bars represent the adhered culture volume of tetracycline resistant XL1-blue E. coli. The grey bars represent the adhered culture volume of GFP expressing, ampicillin resistant CL182 E. coli. Shown is the mean +/−the standard deviation (SD) with n=4.

FIG. 18 illustrates the discrimination of GFP expressing and non-expressing cells. The volume of adhered biomass that is fluorescing green is compared to the amount that is both blue and green and found to be the same. The empty bars show the mean green fluorescence of the adhered culture volume, the hashed bars show total blue fluorescence of the adhered culture volume and the grey bars show the volume of adhered culture where blue and green fluorescence was found to overlap. This shows that the analysis correctly colocalised the green fluorescence with the Hoechst 33342 stained bacteria. Shown is the mean +/−the standard deviation (SD) with n=4.

### DETAILED DESCRIPTION OF THE INVENTION

The present invention can image fluorescent signals from the confocal plane of biofilm cells in the presence of unbound fluorophore or in the presence of intrinsically fluorescent chemical compounds, including potential drug candidates. These assays may make use of any known fluorophore or fluorescent label including but not limited to fluorescein, rhodamine, Texas Red, Amersham Biosciences stains Cy3, Cy5, Cy5.5 and Cy7, Hoechst's nuclear stains and Coumarin stains. (cf. Haugland R. P. Handbook of Fluorescent Probes and Research Chemicals Ed., 1996, Molecular Probes, Inc., Eugene, Oreg.).

Optical Configuration

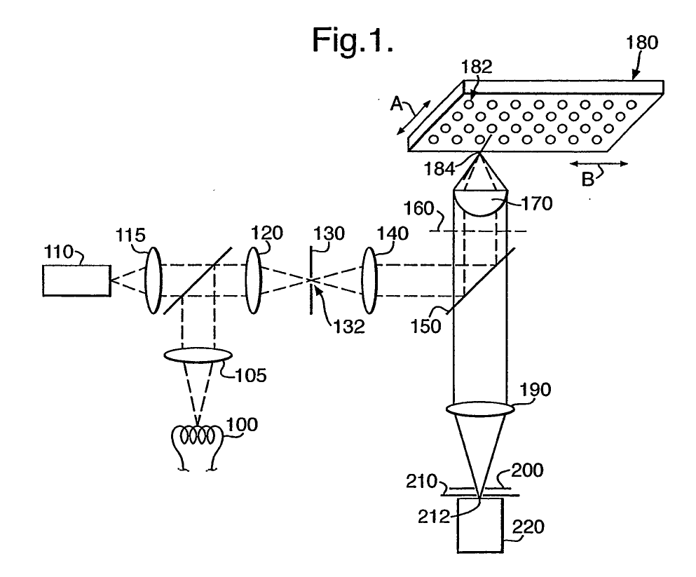

FIG. 1 shows a first embodiment of the present invention. The microscope comprises a source 100 or 110 of electromagnetic radiation for example, in the optical range, 350-750 nm, a cylindrical lens 120, a first slit mask 130, a first relay lens 140, a dichroic mirror 150, an objective lens 170, a microtitre plate 180 containing a two-dimensional array of sample wells 182, a tube lens 190, a filter 200, a second slit mask 210 and a detector 220. These elements are arranged along optical axis OA with slit apertures 132, 212 in masks 130, 210 extending perpendicular to the plane of FIG. 1. The focal lengths of lenses 140, 170 and 190 and the spacings between these lenses as well as the spacings between mask 130 and lens 140, between objective lens 170 and microtitre plate 180 and between lens 190 and mask 210 are such as to provide a confocal microscope. In this embodiment, electromagnetic radiation from a lamp 100 or a laser 110 is focused to a line using a cylindrical lens 120. The shape of the line is optimised by a first slit mask 130.

The slit mask 130 is depicted in an image plane of the optical system that is in a plane conjugate to the object plane. The illumination stripe formed by the aperture 132 in the slit mask 130 is relayed by lens 140, dichroic mirror 150 and objective lens 170 onto a microtitre plate 180 which contains a two-dimensional array of sample wells 182. For convenience of illustration, the optical elements of FIG. 1 are depicted in cross-section and the well plate in perspective. The projection of the line of illumination onto well plate 180 is depicted by line 184 and is also understood to be perpendicular to the plane of FIG. 1. As indicated by arrows A and B, well plate 180 may be moved in two dimensions (X, Y) parallel to the dimensions of the array by means not shown.

In an alternative embodiment, the slit mask 130 resides in a Fourier plane of the optical system that is in a plane conjugate to the objective back focal plane (BFP) 160.

In this case the aperture 132 lies in the plane of the figure, the lens 140 relays the illumination stripe formed by the aperture 132 onto the back focal plane 160 of the objective 170 which transforms it into a line 184 in the object plane perpendicular to the plane of FIG. 1.

In an additional alternative embodiment the slit mask 130 is removed entirely. According to this embodiment, the illumination source is the laser 110, the light from which is focused into the back focal plane 160 of the objective 170. This can be accomplished by the combination of the cylindrical lens 120 and the spherical lens 140 as shown in FIG. 1, or the illumination can be focused directly into the plane 160 by the cylindrical lens 120.

An image of the sample area, for example a sample in a sample well 182, is obtained by projecting the line of illumination onto a plane within the sample, imaging the fluorescence emission therefrom onto a detector 220 and moving the plate 180 in a direction perpendicular to the line of illumination, synchronously with the reading of the detector 220. In the embodiment depicted in FIG. 1, the fluorescence emission is collected by the objective lens 170, projected through the dichroic beamsplitter 150, and imaged by lens 190 through filters 200 and a second slit mask 210 onto a detector 220, such as is appropriate to a confocal imaging system having an infinity-corrected objective lens 170. The dichroic beamsplitter 150 and filter 200 preferentially block light at the illumination wavelength.

The detector 220 illustratively is a camera and may be either one dimensional or two dimensional. If a one dimensional detector is used, slit mask 210 is not needed. The illumination, detection and translation procedures are continued until the prescribed area has been imaged. Mechanical motion is simplified if the sample is translated at a continuous rate. Continuous motion is most useful if the camera read-time is small compared to the exposure-time. In a preferred embodiment, the camera is read continuously. The displacement d of the sample during the combined exposure-time and read- time may be greater than or less than the width of the illumination line W, for example 0.5W≦d≦5W. All of the wells of a multiwell plate can be imaged in a similar manner.

Alternatively, the microscope can be configured to focus a line of illumination across a number of adjacent wells, limited primarily by the field-of-view of the optical system. Finally, more than one microscope can be used simultaneously. The size and shape of the illumination stripe 184 is determined by the width and length of the Fourier transform stripe in the objective lens back focal plane 160. For example, the length of the line 184 is determined by the width of the line in 160 and conversely the width in 184 is determined by the length in 160. For diffraction-limited performance, the length of the illumination stripe at 160 is chosen to overfill the objective back aperture. It will be evident to one skilled in the art that the size and shape of the illumination stripe 184 can be controlled by the combination of the focal length of the cylindrical lens 120 and the beam size at 120, that is by the effective numerical aperture in each dimension, within the restrictions imposed by aberrations in the objective, and the objective field of view.

The dimensions of the line of illumination 184 are chosen to optimise the signal to noise ratio. Consequently, they are sample dependent. Depending on the assay, the resolution may be varied between diffraction-limited and approximately 5 μm. The beam length is preferably determined by the objective field of view, exemplarily between 0.5 and 1.5 mm. A Nikon ELWD, 0.6 NA, 40× objectives, for example, has a field of view of approximately 0.75 mm. The diffraction-limited resolution for 633 nm radiation with this objective is approximately 0.6 μm, or approximately 1100 resolution elements.

The effective depth resolution is determined principally by the width of aperture 212 in slit mask 210 or the width of the one dimensional detector and the image magnification created by the combination of the objective lens 170 and lens 190. The best axial resolution of a confocal microscope approaches 1 μm. The Confocal Handbook (J. B. Pawley, Editor, Handbook of Biological Confocal Microscopy, 2nd Edition, Plenum, New York, 1995) gives several expressions for the axial resolution, d_{z}, but two in particular are most relevant:

$${d}_{z} = \frac{1.77 \cdot λ}{{( NA )}^{2}}$$

$${d}_{z} = \frac{0.22 \cdot λ}{n \cdot {sin}^{2} ( \frac{α}{2} )}$$

where λ is the wavelength of the light, NA is the numerical aperture of the microscope objective, n is the refractive index of the medium and a refers to the angle used to compute the NA. Both formulas assume ideal or zero-size pinholes. The first equation applies the Rayleigh criterion along the optic axis (this direction is generally referred to as the z-direction, while the plane perpendicular to the optic axis is the x-y plane). A microscope objective focused in air will produce a 3D diffraction-limited spot with an Airy disk cross-section at the focal point in the x-y plane and another distribution along the x-z or y-z plane. This equation best describes the axial resolution in photoluminescence imaging. The second equation is derived using paraxial (small angle) theory assuming the object being viewed is a perfect planar mirror. At low NA the first equation agrees with the second equation except for a factor of two due to specular reflection. These equations should be considered only approximations which in some circumstances will fail to predict resolutions accurately.

It is usually preferable to determine the effective depth resolution experimentally. For example, it can be done by using as a sample a fluorescent polystyrene bead with a diameter smaller than the resolution limit of the microscope. The image of the this object obtained by stepping the focal plane through the bead is therefore the image of a sub-resolution object and can be used as a definition of the Point Spread Function of the optical system. The full width at half maximum in the Z direction (axial direction) is the used as a definition of the axial resolution of the optical system.

The effective numerical aperture (“NA”) of the illumination is less than the NA of the objective. The fluorescence emission is, however, collected with the full NA of the objective lens. The width of aperture 212 must be increased so as to detect emission from the larger illumination volume. At an aperture width a few times larger than the diffraction limit, geometrical optics provides an adequate approximation for the size of the detection-volume element:
Lateral Width: a_{d}=d_{d}/m,
Axial Width: z_{d},=√2a_{d}/tan α,
where m is the magnification, d_{d} is the width of aperture 212 and α is the half-angle subtended by the objective 170. It is an important part of the present invention that the illumination aperture 132 or its equivalent in the embodiment having no aperture and the detection aperture 212 be independently controllable.
Multi-Wavelength Configuration

An embodiment enabling multi-wavelength fluorescence imaging is preferred for certain types of assays. It is generally advantageous and often necessary that two or more measurements be made simultaneously since one important parameter in a biological response is time.

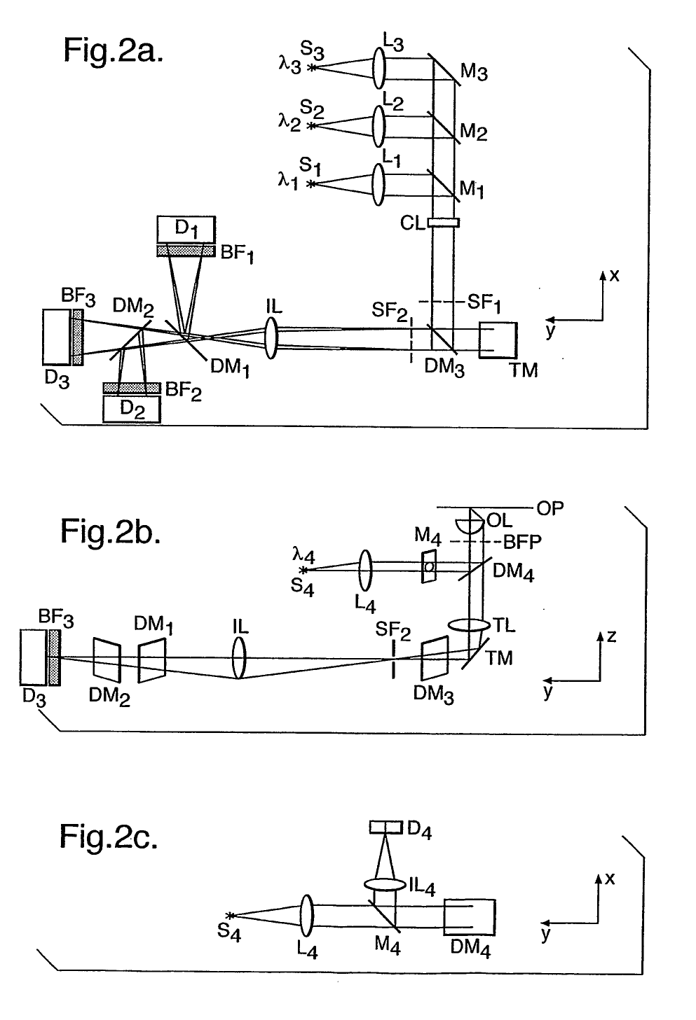

The number of independent wavelengths or colours will depend on the specific assay being performed. In one embodiment three illumination wavelengths are used. FIGS. 2(a) and 2(b) depict the ray paths in a three-colour line- scan confocal imaging system, from a top view and a side view respectively. In general, the system comprises several sources Sₙ of electromagnetic radiation, collimating lenses Lₙ, and mirrors Mₙ for producing a collimated beam that is focused by cylindrical lines CL into an elongated beam at first spatial filter SF₁, a confocal microscope between first spatial filter SF₁, and second spatial filter SF₂ and an imaging lens IL, beamsplitters DM₁, and DM₂ and detectors Dn for separating and detecting the different wavelength components of fluorescent radiation from the sample. Spatial filters SF₁ and SF₂, preferably are slit masks.

In particular, FIG. 2(a) depicts sources, S₁, S₂ and S₃ for colours λ₁, λ₂ and λ₃, and lenses L₁, L₂ and L₃ that collimate the light from the respective sources. Lenses L₁, L₂ and L₃, preferably are adjusted to compensate for any chromaticity of the other lenses in the system. Mirrors M₁, M₂ and M₃ are used to combine the illumination colours from sources Sn. The mirrors M₁ and M₂, are partially transmitting, partially reflecting and preferentially dichroic. M₂ for example, should preferentially transmit λ₃, and preferentially reflect λ₂. It is thus preferential that λ₃ be greater than 2. Operation of the microscope in a confocal mode requires that the combined excitation beams from sources Sn be focused to a “line”, or a highly eccentric ellipse, in the object plane OP. As discussed in connection with FIG. 1 above, a variety of configurations may be used to accomplish this. In the embodiment depicted in FIG. 2, the combined illumination beams are focused by cylindrical lens CL into an elongated ellipse that is coincident with the slit in the spatial filter SF₁. As drawn in FIGS. 2a and 2b, the slit mask SF₁, resides in an image plane of the system, aligned perpendicular to the propagation of the illumination light and with its long axis in the plane of the page of FIG. 2a. The lenses TL and OL relay the illumination line from the plane containing SF₁, to the object plane OP. A turning mirror, TM, is for convenience. In another embodiment, DM₃ is between TL and OL and CL focuses the illumination light directly into the BFP. Other embodiments will be evident to one skilled in the art.

Referring to FIG. 2(b), the light emitted by the sample and collected by the objective lens OL is imaged by the tube lens TL onto the spatial filter SF₂. SF₂ is preferentially a slit aligned so as to extend perpendicular to the plane of the page. Thus, the light passed by filter SF₂ is substantially a line of illumination. SF₂ may be placed in the primary image plane or any plane conjugate thereto. DM₃ is partially reflecting, partially transmitting and preferably “multichroic”. Multi-wavelength “dichroic” mirrors or “multichroic” mirrors can be obtained that preferentially reflect certain wavelength bands and preferentially transmit others.

Herein, δλ₁ will be defined to be the fluorescence emission excited by λ₁. This will, in general, be a distribution of wavelengths somewhat longer than λ₁, and δλ₂ and δλ₃ are defined analogously. DM₃ preferentially reflects λₙ, and preferentially transmits δλₙ where n=1,2,3. The light transmitted by SF₂ is imaged onto the detection devices, which reside in planes conjugate to the primary image plane. In FIG. 2(a), an image of the spatial filter SF₂ is created by lens IL on all three detectors, Dₙ. This embodiment is preferred in applications requiring near-perfect registry between the images generated by the respective detectors. In another embodiment, individual lenses ILₙ, are associated with the detection devices, the lens pairs IL and ILₙ, serving to relay the image of the spatial filter SF₂ onto the respective detectors Dₙ. The light is split among the detectors by mirrors DM₁ and DM₂. The mirrors are partially transmitting, partially reflecting, and preferentially dichroic. DM₁, preferentially reflects δλ₁, and preferentially transmits δλ₂ and δλ₃. The blocking filter, BF₁, preferentially transmits δλ₁, effectively blocking all other wavelengths present. DM₂ preferentially reflects δλ₂ and preferentially transmits δλ₃. The blocking filters, BF₂ and BF₃, preferentially transmit δλ₂ and δλ₃ respectively, effectively blocking all other wavelengths present.

Autofocus

According to this embodiment of present invention, the sample lies in each of a plurality of planes which can be moved, by a scanning mechanism, into the object plane of an imaging system. Accordingly, the invention provides an autofocus mechanism that maintains the currently selected plane of the sample in the field-of-view of the imaging system within the object plane of that system. The precision of planarity is determined by the depth-of-field of the system. In a preferred embodiment, the depth-of-field is approximately 10 μm and the field-of-view is approximately 1 μmm².

The autofocus system allows precise movement of the objective and thus the focal plane. The autofocus system operates with negligible delay, that is, the response time is short relative to the image acquisition-time, for example 0.01-0.1 s. In addition, the autofocus light source is independent of the illumination light sources and the sample properties. Among other advantages, this configuration permits the position of the sample carrier along the optical axis of the imaging system to be determined independent of the position of the object plane.

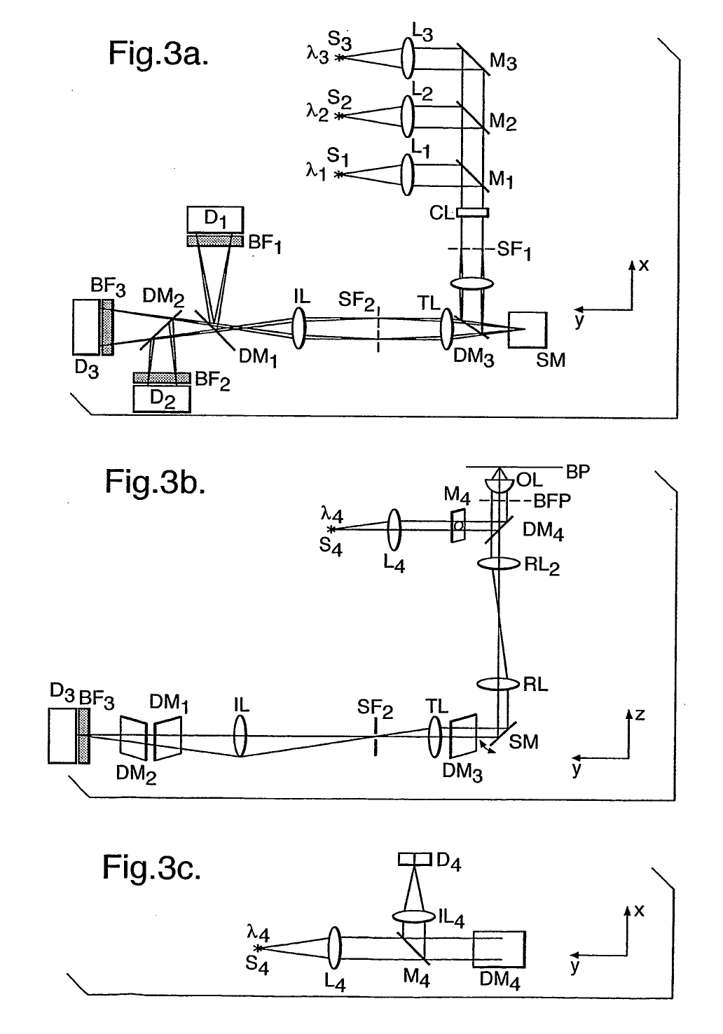

One embodiment of a single-beam autofocus is provided in FIGS. 2 and 3, where a separate light source S₄ of wavelength λ₄, and detector D₄ are shown. The wavelength λ₄ is necessarily distinct from the sample fluorescence, and preferentially a wavelength that cannot excite appreciable fluorescence in the sample. Thus, λ₄ is preferentially in the near infrared, exemplarily 800-1000 mn. The partially transmitting, partially reflecting mirror, DM₄ is preferentially dichroic, reflecting λ₄ and transmitting λₙ, and δλₙ, where n=1,2,3. Optically-based autofocus mechanisms suitable for the present application are known. For example, an astigmatic-lens-based system for the generation of a position error signal suitable for servo control is disclosed in Applied Optics 23, 565-570 (1984). A focus error detection system utilizing a “skew beam” is disclosed in SPIE 200, 73-78 (1979). The latter approach is readily implemented according to FIGS. 2 and 3, where D₄ is a split detector.

For use with a microtitre plate having a biofilm residing on the well bottom, the servo loop must, however, be broken to move between wells. This can result in substantial time delays because of the need to refocus each time the illumination is moved to another well.

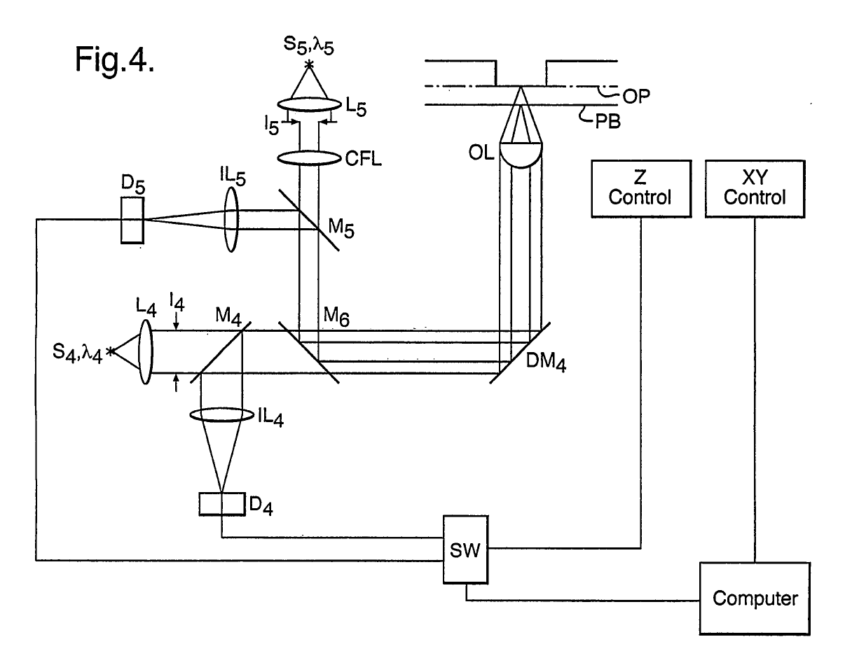

Continuous closed-loop control of the relative position of the sample plane and the object plane is provided in a preferred embodiment of the present invention, depicted in FIG. 4. This system utilises two independent beams of electromagnetic radiation. One, originating from S₅, is focused on the continuous surface, exemplarily the bottom of a microtitre plate. The other, originating from S₄ is focused on the discontinuous surface, for example, the well bottom of a microtitre plate. In one embodiment, the beams originating from S₄ and S₅ have wavelengths λ₄ and λ₅, respectively. The beam of wavelength λ₄ is collimated by L₄, apertured by iris I₄, and focused onto the discontinuous surface by the objective lens OL.

The beam of wavelength 5 is collimated by L₅, apertured by iris I₅, and focused onto the continuous surface by the lens CFL in conjunction with the objective lens OL. The reflected light is focused onto the detectors D₄ and D₅, by the lenses IL₄ and IL₅, respectively. The partially transmitting, partially reflecting mirror, DM₄ is preferentially dichroic, reflecting λ₄ and λ₅ and transmitting λₙ and δλₙ, where n=1,2,3. The mirrors, M₄, M₅ and M₆, are partially transmitting, partially reflecting. In the case that λ₄ and λ₅ are distinct, M₆ is preferentially dichroic.

According to the embodiment wherein the biofilm resides in a microtitre plate, λ₄ is focused onto the well bottom. The object plane can be offset from the well bottom by a variable distance. This is accomplished by adjusting L₄ or alternatively by an offset adjustment in the servo control loop. For convenience of description, it will be assumed that λ₄ focuses in the object plane.

The operation of the autofocus system is as follows. If the bottom of the sample well is not in the focal plane of objective lens OL, detector D₄ generates an error signal that is supplied through switch SW to the Z control. The Z control controls a motor (not shown) for moving the microtitre plate toward or away from the objective lens.

Pseudo-closed loop control is provided in the preferred embodiment of single-beam autofocus which operates as follows. At the end of a scan the computer terminal operates SW to switch control to a sample-and-hold device which maintains the Z control output at a constant level while the plate is moved on to the next well after which SW is switched back to D₄.

Detection Devices

A feature of the disclosed apparatus is the use of a detection device having manifold, independent detection elements in a plane conjugate to the object plane. As discussed above, line illumination is advantageous principally in applications requiring rapid imaging. The potential speed increase inherent in the parallelism of line illumination as compared to point illumination is, however, only realised if the imaging system is capable of detecting the light emitted from each point of the sample along the illumination line, simultaneously.

One embodiment uses a continuous-read line-camera, and in a preferred embodiment a rectangular CCD is used as a line-camera. Both embodiments have no dead-time between lines within an image or between images. An additional advantage of the present invention is that a larger effective field-of-view is achievable in the stage- scanning embodiment, discussed below.

The properties required of the detection device can be further clarified by considering the following preferred embodiment. The resolution limit of the objective lens is <1 μm, typically ˜0.5 μm, and the detector comprises an array of ˜1000 independent elements. Resolution, field-of-view (FOV) and image acquisition-rate are not independent variables, necessitating compromise among these performance parameters. In general, the magnification of the optical system is set so as to image as large a FOV as possible without sacrificing resolution. For example, a ˜1 mm field-of-view could be imaged onto a 1000-element array at 1 μm pixelation. If the detection elements are 20 μm square, then the system magnification would be set to 20×. Note that this will not result in 1 μm resolution.

Pixelation is not equivalent to resolution. If, for example, the inherent resolution limit of the objective lens is 0.5 μm and each 0.5 μm×0.5 μm region in the object plane is mapped onto a pixel, the true resolution of the resulting digital image is not 0.5 μm. To achieve true 0.5 μm resolution, the pixelation would need to correspond to a region ˜0.2 μm×0.2 in the object plane. In one preferred embodiment, the magnification of the imaging system is set to achieve the true resolution of the optics.

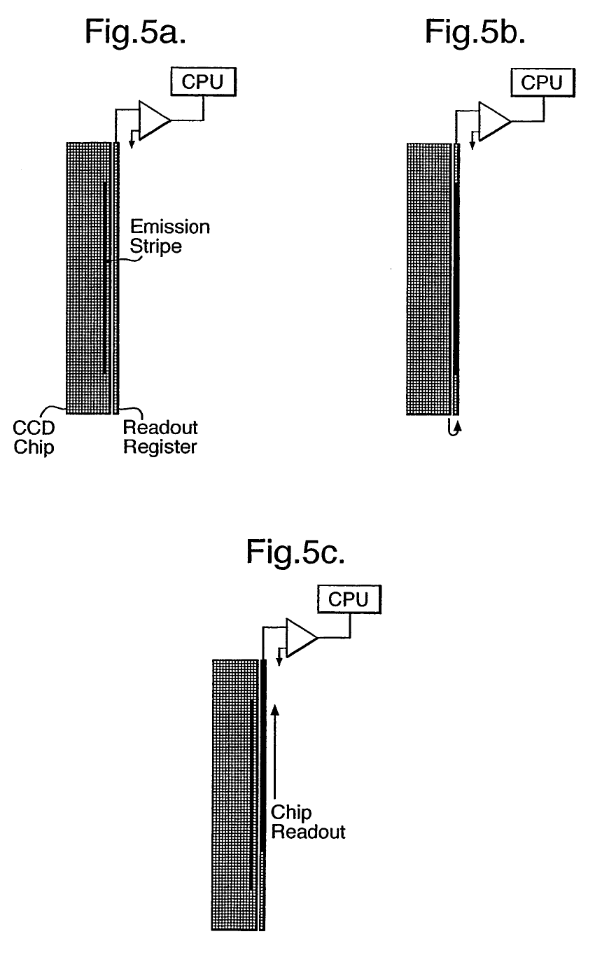

Preferably, for high detection efficiency, low noise and sufficient read-out speed, the detectors used are CCD cameras. In FIG. 5, a rectangular CCD camera is depicted having an m×n array of detector elements where m is substantially less than n. The image of the fluorescence emission covers one row that is preferably proximate to the read register. This miniinses transfer time and avoids accumulating spurious counts into the signal from the rows between the illuminated row and the read-register.

In principle, one could set the magnification of the optical system so that the height of the image of the slit SF₂ on the CCD camera is one pixel, as depicted in FIG. 5.

In practice, it is difficult to maintain perfect alignment between the illumination line and the camera row-axis, and even more difficult to maintain alignment among three cameras and the illumination in the multi-wavelength embodiment as exemplified in FIGS. 2 and 3. By binning together a few of the detector elements, exemplarily two to five, in each column of the camera the alignment condition can be relaxed while suffering a minimal penalty in read-noise or read-time.

An additional advantage of the preferred embodiment having one or more rectangular CCD cameras as detection devices in conjunction with a variable-width detection spatial filter, SF₂ in FIGS. 2 and 3 and 210 in FIG. 1, each disposed in a plane conjugate to the object plane, is elucidated by the following. As discussed above, in one embodiment of the present invention the detection spatial filter is omitted and a line-camera is used as a combined detection spatial filter and detection device. But as was also discussed above, a variable-width detection spatial filter permits the optimisation of the detection volume so as to optimise the sample-dependent signal-to-noise ratio. The following preferred embodiment retains the advantage of a line-camera, namely speed, and the flexibility of a variable detection volume. The magnification is set so as to image a diffraction-limited line of height h onto one row of the camera. The width of the detection spatial filter d is preferably variable, with:
h≦d≦10h.

The detectors in the illuminated columns of the camera are binned, prior to reading, which is an operation that requires a negligible time compared to the exposure- and read-times.

In one preferred embodiment, the cameras are Princeton Instruments NTE/CCD-1340/100-EMD. The read-rate in a preferred embodiment is 1 MHz at a few electrons of read-noise. The pixel format is 1340×100, and the camera can be wired to shift the majority of the rows (80%) away from the region of interest, making the camera effectively 1340×20.

In addition to the above mentioned advantage of a continuous read camera, namely the absence of dead-time between successive acquisitions, an additional advantage is that it permits the acquisition of rectangular images having a length limited only by the extent of the sample. The length is determined by the lesser of the camera width and the extent of the line illumination. In a preferred embodiment the attached biofilm is disposed on the bottom of a well in a 96-well microtitre plate, the diameter of which is 7 mm. A strip 1 μm×1 mm is illuminated and the radiation emitted from the illuminated area is imaged onto the detection device. The optical train is designed such that the field-of-view is ˜1 mm². An image of the well-bottom can be generated at 1 μm pixelation over a 1×7 mm field.

Environmental Control

In an embodiment of the present invention, assays are performed on a living biofilm. Live-cell assays frequently require a reasonable approximation to physiological conditions to run properly. Among the important parameters is temperature. It is desirable to incorporate a means to raise and lower the temperature, in particular, to maintain the temperature of the sample at 37° C. In another embodiment, control over relative humidity, and/or CO₂ and/or O₂ is necessary to maintain the viability of the living biofilm. In addition, controlling humidity to minimise evaporation is important for small sample volumes.

Three embodiments providing a microtitre plate at an elevated temperature, preferably 37° C., compatible with the confocal imaging system follow.

The imaging system preferably resides within a light-proof enclosure. In a first embodiment, the sample plate is maintained at the desired temperature by maintaining the entire interior of the enclosure at that temperature. At 37° C., however, unless elevated humidity is purposefully maintained, evaporation cooling will reduce the sample volume limiting the assay duration.

A second embodiment provides a heated cover for the microwell plate which allows the plate to move under the stationary cover. The cover has a single opening above the well aligned with the optical axis of the microscope. This opening permits dispensing into the active well while maintaining heating and limited circulation to the remainder of the plate. A space between the heated cover plate and microwell plate of approximately 0.5 mm allows free movement of the microwell plate and minimises evaporation. As the contents of the interrogated well are exposed to ambient conditions though the dispenser opening for at most a few seconds, said contents suffer no significant temperature change during the measurement.

In a third embodiment, a thin, heated sapphire window is used as a plate bottom enclosure. A pattern of resistive heaters along the well separators maintain the window temperature at the desired level.

In additional embodiments, the three disclosed methods can be variously combined.

Integrated Dispenser

One embodiment of the present invention provides an integrated dispenser. For assays run in 96- or 384-well plates, addition volumes in this range 20-100 μL are desirable A single head dispenser, as is appropriate, for example, to the addition of an agonist of ion-channel activity, is the IVEK Dispense 2000. Comparable units are available from CAVRO. More generally, it is desirable to be able to dispense a unique compound into each well. One embodiment provides a single head dispenser on a robotic motion device that shuttles the dispense head between the analysis station, the source plate containing the unique compounds and the tip cleansing station. The latter is a wash station for a fixed tip dispenser and a tip changing station for a disposable tip dispenser. This system provides the desired functionality relatively inexpensively, but it is low throughput, requiring approximately 30 seconds per compound aspiration-dispense-cleanse cycle. An alternative embodiment is provided by integrating a multi-head dispenser such as the Hamilton Microlab MPH-96 into the confocal imaging system. The MPH-96 consists of 96 independent fixed tip dispensers mounted to a robotic motion device capable of executing the aspirate-dispense-wash cycle described above.

In an additional preferred embodiment of the invention, employed in automated screening assays, the imaging system is integrated with plate-handling robots, such as the Zymark Twister.

Assays

Numerous variations of the assay methods described below in Examples 1-3 can be practised in accordance with the invention. In general, a characteristic intensity, and/or spatial distribution, and/or temporal distribution of one or more fluorescently-labelled species are used to quantify the assay.

Data Processing System

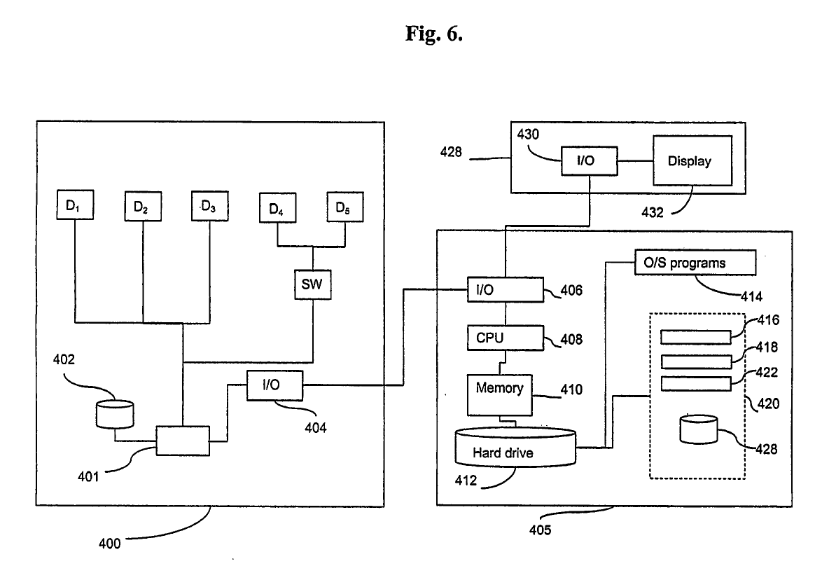

FIG. 6 shows a schematic illustration of data processing components of a system arranged in accordance with the invention. The system, based on the Amersham Biosciences IN Cell Analyzerm system, includes a confocal microscope 400 as described above, which includes the detectors D₁, D₂, D₃, D₄, D₅, the switch SW, a control unit 401, an image data store 402 and an Input/Output (I/O) device 404. Note that alternative arrangements are possible, including an arrangement in which D₄ and D₅ are omitted, an arrangement in which D₄ is omitted and an arrangement in which D₅ is omitted. The image data store 402 may be any suitable form of storage device or may alternatively be omitted, with the output data being transmitted to be stored on the associated computer terminal 405. The associated computer terminal 405 includes a central processing unit (CPU) 408, memory 410, a data storage device such as a hard disc drive 412 and I/O devices 406 which facilitate interconnection of the computer with the microscope 400 and the computer with a display element 432 of a screen 428 via a screen I/O device 430, respectively. Operating system programs 414 are stored on the hard disc drive 412, and control, in a known manner, low level operation of the computer terminal 405. Program files and data 420 are also stored on the hard disc drive 412, and control, in a known manner, outputs to an operator via associated devices and output data stored on the hard disc drive. The associated devices include a display 432 as an element of the screen 428, a pointing device (not shown) and keyboard (not shown), which receive input from, and output information to, the operator via further I/O devices (not shown). Included in the program files 420 stored on the hard drive 412 are an image processing and analysis application 416, an assay control application 418, and a database 422 for storing image data received from the microscope 400 and output files produced during data processing. The image processing and analysis application 418 may be a customized version of known image processing and analysis software packages, such as Image-Pro™ from Media Cybernetics.

The performance of an assay using the confocal microscope 400 is controlled using control application 418, and the image data are acquired. After the end of acquisition of image data for at least one well in a microtiter plate by at least one detector D₁, D₂, D₃, the image data are transmitted to the computer 405 and stored in the database 422 on the computer terminal hard drive 412, at which point the image data can be processed using the image processing and analysis application 416, as will be described in greater detail below.

Data Analysis

In general the acquisition and analysis of the data comprises a number of discrete steps. The fluorescence is converted into one or more digital images in which the digital values are proportional to the intensity of the fluorescent radiation incident on each pixel of the detection device. Within this step a correction is made for the non-uniform response of the imaging system across the field of view wherein the background-subtracted data are divided by a so-called flat-field file.

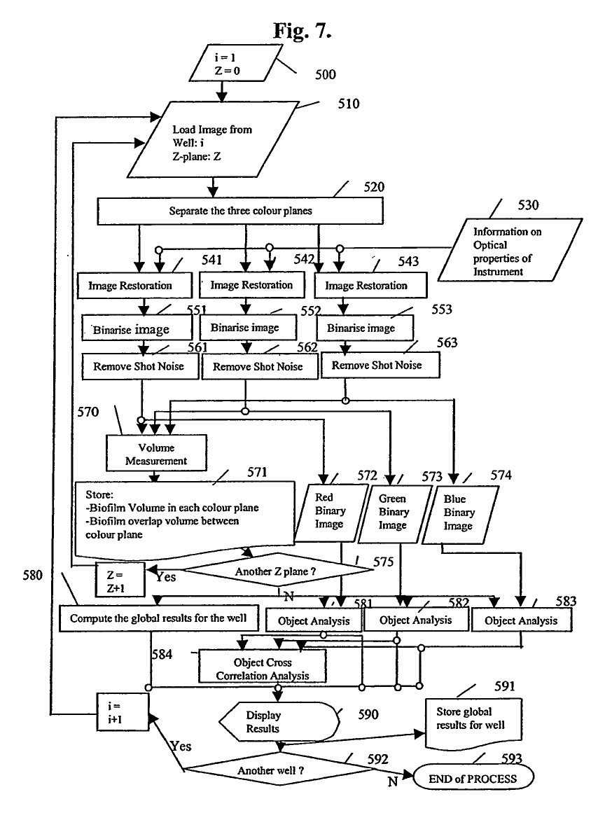

Data analysis is performed as described in FIGS. 7 to 14 by the image processing and analysis application (416). FIG. 7 represents the flow diagram of the analysis algorithm. The analysis starts (500) with the first Z-plane (Z=1) of the first well (i=1). The image of the first Z-plane of the first well is loaded into the memory of the computer (510). The image can be composed of one or more colour planes (for example, Red (R), Green (G) and Blue (B)) corresponding to the images recorded by the three cameras of the instrument. The information from the three colour planes is separated (520) into three images and each image is processed separately. All three images are processed in the same manner. Firstly, the image is restored to compensate for the distortion and aberration introduced by the optics (Red image in process (541); green image in process (542) and blue image in process (543)). The image restoration could for example be a deconvolution or any other image restoration technique well known in the art. The image restoration techniques usually require the input of some information (530) on the optical properties of the imaging system. For example, in the case of the deconvolution technique, the Point Spread Function (PSF) of the optical system may be required.

Secondly, each image is thresholded in an image data thresholding step of the image data processing algorithm. In this embodiment the thresholding step involves binarising each image (process (551), (552) and (553)) in order to convert the grey scale image into a binary image where, for example, 1 represents image pixels occupied by micro-organisms in the biofilm and 0 represents image pixels occupied by free space between micro-organisms. In this embodiment of the invention, the value of the binarisation threshold is determined without user intervention. The value of the binarisation threshold is determined for each image separately. Alternatively, a single threshold may be calculated and applied to all the images in each well, namely the stack of images in different planes, for each of the colour channels separately.

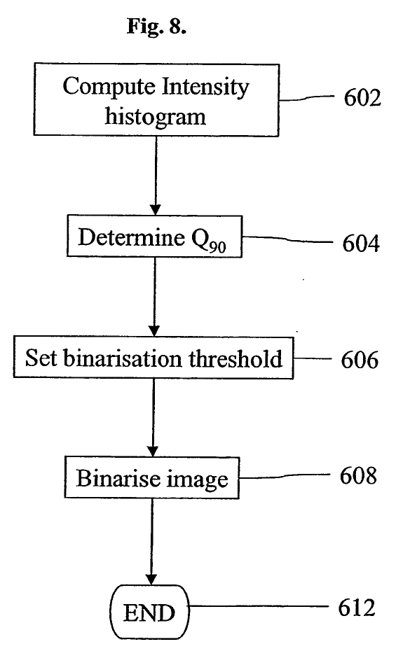

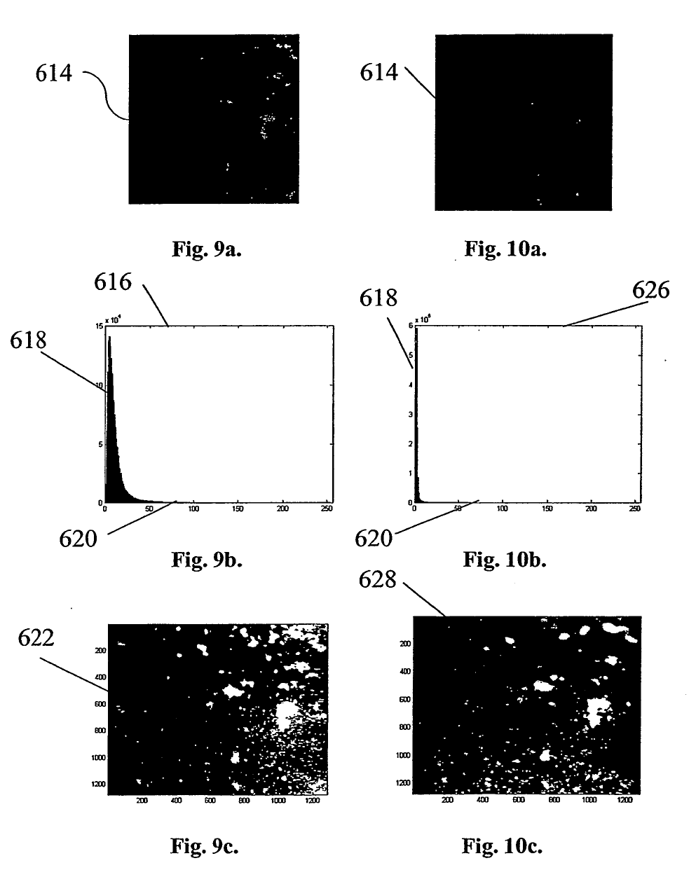

FIG. 8 is a flow chart of an image data binarising process (process 551, 552, and 553) of the analysis algorithm. FIGS. 9(a)-(c) illustrate stages of the binarising process (551) for a red colour plane image and FIGS. 10(a)-(c) illustrate stages of the binarising process (552) for a green colour plane image.

FIG. 9(a) shows the red (R) colour plane image (614), intensity values of which are analysed in the binarising process, in this embodiment by computing (602) an intensity histogram (616), as illustrated by FIG. 9(b). The intensity histogram (616) plots a number of pixels of the image having a specific intensity on a first axis (618) against an intensity of the red image (614) on a second axis (620). Using the intensity histogram (616), the intensity value below which the intensity values of a predetermined proportion of the analysed set of pixels is determined (604). This predetermined quantile is envisaged to have a value within the range 70%-95%, preferably within the range 85%-95% and more preferably of the approximate value 90% (Q₉₀). The 90% quantile (Q₉₀) represents an intensity value below which 90% of the total number of pixels of the image (614) lie. The intensity represented by the 90% quantile (Q₉₀) is used to set an optimised binarisation threshold TR (606) for the red image (614). In this example the binarisation threshold TR is an intensity value of 24.

The red image (614) is then binarised (608) using the threshold (TR) to produce a binary image. In the mask, any pixel of the red image (614) having an intensity greater than the threshold (T_{R}) is set to 1 and any pixel of the red image (614) having an intensity equal to or lower than the threshold (T_{R}) is set to 0. A pixel set to 1 generally corresponds to an element of a bacterium in the red image (614) and a pixel set to 0 corresponds to a background element in the red image (614). FIG. 9(c) illustrates a red binary image (622) on which pixels set to 1 are shown as white and pixels set to 0 are shown as black. The binarising process (551) then ends (612).

In a similar manner to the application of the binarising step to the red (R) colour plane image (614) as described above, both the green (G) and the blue (B) colour plane images are binarised (552, 553) to form green and blue binary images, using the same predetermined quantile to set the thresholding level. FIG. 10(a) shows the green (G) colour plane image (624). FIG. 10(b) shows an intensity histogram (626) for the green colour plane image (624) which is plotted on the same first and second axes (618, 620) as for the red image (614). In this example the binarisation threshold TG is approximately an intensity value of 4, this value having been optimised for the green colour plane image (624). FIG. 10(c) shows a green binary image (628). Binarised pixels set to 1 are white and binarised pixels set to 0 are black.

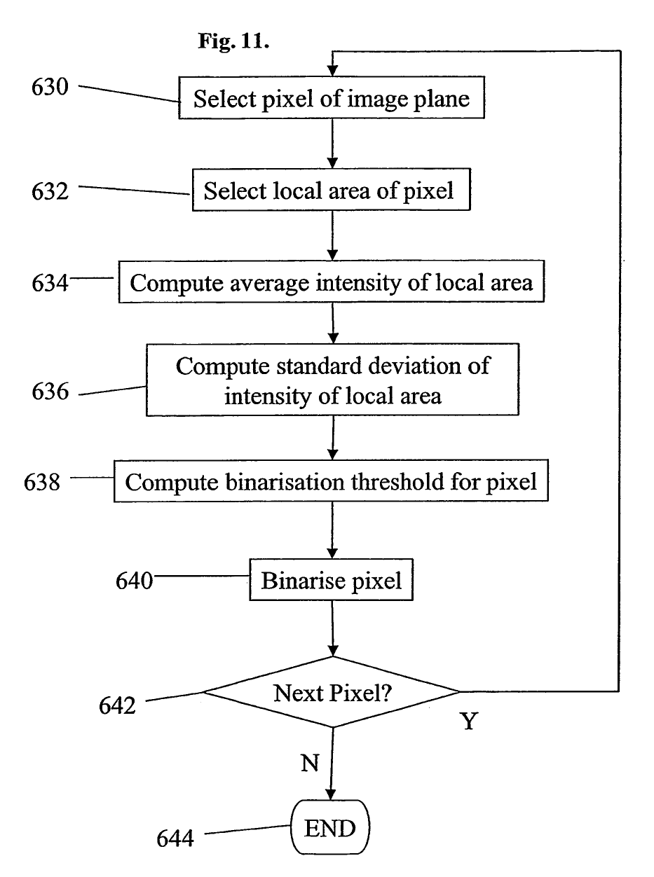

FIG. 11 illustrates an alternative image data binarising process. This alternative binarising step is appropriate for binarising images having a regional variation in intensity. This regional variation intensity may be due to a non-uniform illumination of the plane of the biofilm being scanned, or to a variation in a thickness or a density of the biofilm being scanned. In the instance of a non-uniform illumination it is possible to use an image correction method such as a flatfield correction to minimise the regional variation in intensity.

In the alternative binarising process, one pixel of the image, for example of the red (R) colour plane image (614), is selected (630). A square with a fixed area is centred about the selected pixel and thus selects (632) a local area of the pixel of the image. The area of the square is determined prior to scanning of the sample. For the local area of the selected pixel both an average intensity (Al) is computed (634) and a standard deviation (SD) of the intensity (636) are computed. A pixel binarisation threshold T_{P} is then computed (638). The pixel binarisation threshold (T_{P}) is computed as follows:
T_{P}=AI+(M×SD)
where M is a constant with a preferred value in the range of 0.2 to 4, preferably in the range of 0.5 to 3 and more preferably approximately 1. The value of M is initially the same when the binarising process is applied to other images, for example the green and blue colour plane images.

Next, the selected pixel is binarised (640). If the intensity of the selected pixel has an intensity greater than the pixel threshold (T_{P}) the pixel is set to 1. If the intensity of the selected pixel has an intensity less than the pixel threshold (T_{P}) the pixel is set to 0. A check (642) is performed to determine if there are other pixels of the image (614) which have not been binarised. If there are further pixels which have not been binarised, the thresholding step is repeated by selecting a further, different, pixel of the image plane (630). Following the binarising of every pixel of the image plane, the binarising step ends (644) yielding a binary image.

Thirdly, in the analysis algorithm the shot noise is removed from the images (processes (561), (562) and (563)) by using any of the well known techniques of image analysis (e.g. erosion-dilation, filtering using for example a Gaussian filter). Fourthly, the processed images of the three colour planes are stored in the memory (572), (573),(574). The images for all the colour planes for every Z-plane for a single well are kept in order to do the 3D analysis at a later stage (see processes (581) to (584)). The images are also used as input for the sub-routine (570). The sub-routine (570) determines the volume of the biofilm in each colour plane and the volume of the overlap between colour planes.

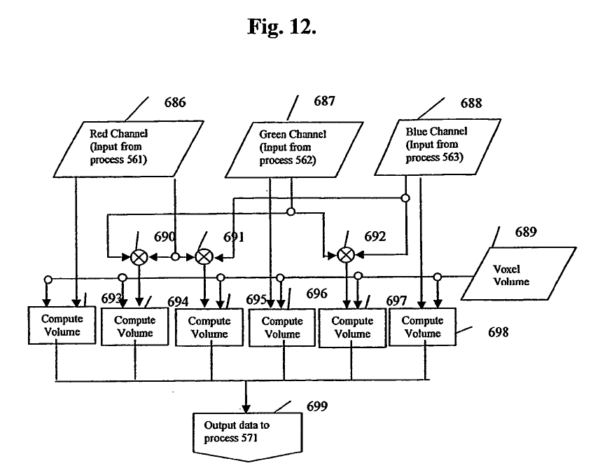

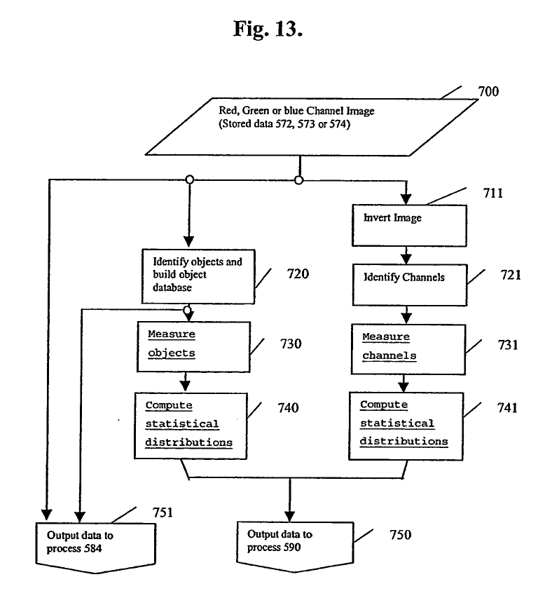

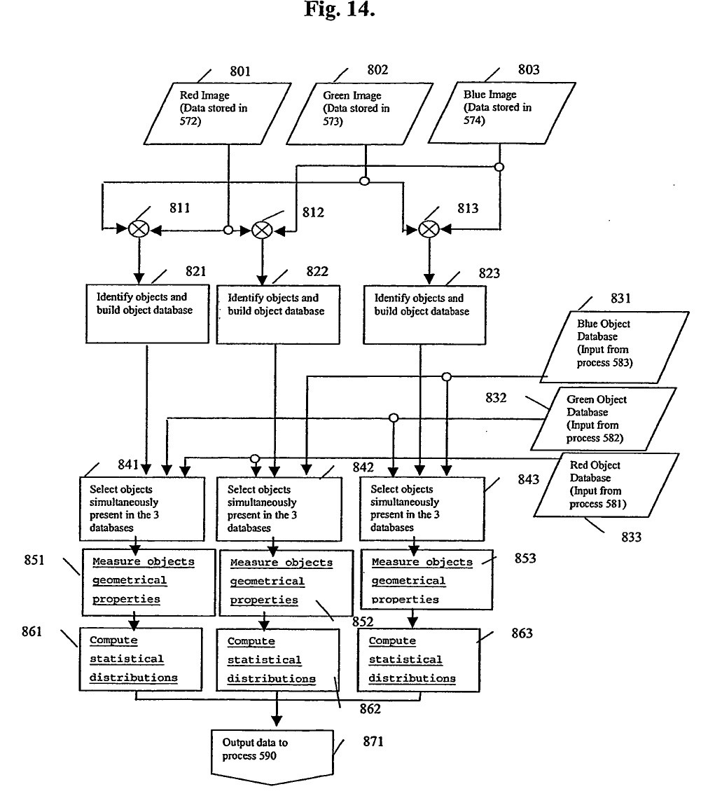

The flow chart for this subroutine is shown in FIG. 12. Fifthly, the program stores the results of the volume measurement to disk (571). Sixthly, the program determines whether there is another Z-plane to be processed for this well (575). If there is one, the whole process is repeated from process (510), using image from the next Z-plane (Z=Z+1). Seventhly, if there is no other Z-plate, the program computes the final results for that well (580). These final results include for example, the total volume of biofilm for the different colour planes or the total volume of free space between micro-organisms. Eighthly the program carries out the 3D object analysis. Each stack of colour planes is processed separately. The same subroutine is used for each colour stack separately. The stacks of images are stored at an earlier stage in the program. Process (581) uses the red stack (572); process (582) uses the green stack (573) and process (583) uses the blue stack (574). This subroutine identifies the clusters of micro-organisms and the channels between the clusters. It also determines the statistical distribution of geometrical shape parameters for the microbial clusters and the channels. The flowchart for the subroutine is shown on FIG. 13 and it is the same flow chart for every colour plane. Ninthly, the results from the 3D object analysis sub-routine are used as input for Object Cross Correlation sub-routine (584). This subroutine identifies any correlation between objects presents in different colour planes. The flow chart for this sub-routine is shown in FIG. 14.

Tenthly, all the results for this well are displayed on screen (590) and are stored on disk (591). Prior to displaying the results on the screen (590), the results are validated in a validation process to determine a quality and therefore a success of the binarisation processes (551, 552, 553) and to ensure that pixels of the images have generally not been incorrectly set to 1 or 0. This could occur if for example the binarisation threshold was not correctly optimised for the image or if further image processing of the binarised images, for example removing shot noise (561, 562, 563), removed in error relatively large numbers of pixels set to 1.

If the validation process determines the binarisation of an image to have been of a poor quality and therefore unsuccessful, the analysis algorithm is repeated for the image. In this repetition of the analysis algorithm the binarisation threshold is varied. Therefore, for the embodiment where the a predetermined quantile is used to determine the binarisation threshold, the percentage of the quantile is modified by a stepwise increase or decrease as desired. For the alternative binarising step, the value of the constant M is similarly modified in the repeat analysis. Following the repetition of the analysis algorithm, the results are again validated by the validation process to determine the success of the binarisation. If the results of the binarisation are again considered to be of a poor quality and therefore unsuccessful the analysis algorithm is again repeated, with a further modification of the percentage of the quantile, or the value of M, as appropriate. Once the validation process considers the results of the binarisation to have been successful, the results are displayed on the screen (590).

The validation process may involve one or more steps, which will be described below. An example of a step which may be carried out in the validation process involves checking a that no greater than a predetermined proportion, for example approximately 80%, of pixels of the final processed image of the analysis algorithm that are set to 1. If greater than the predetermined proportion of the pixels are set to 1, the validation process indicates that the threshold value was set too low. Consequently the results are considered unsuccessful and the analysis algorithm is repeated for the image with a higher threshold value. In the case where the binarisation threshold was set using the 90% quantile (Q₉₀) it was described that therefore approximately 90% of the pixels having the lowest intensities of the image would be set to 0. Further processing of the binarised image, including removing shot noise, would set a small proportion of pixels from 1 to 0 and therefore it would be expected that a proportion somewhat less than approximately 10% of the pixels would be set to 1. If in the validation process it is found that greater than approximately 80% of the pixels are set to 1 then this identifies that the binarisation threshold value should be modified and the analysis algorithm of the image repeated.

A further example of a step which may be carried out in the validation process involves checking whether less than a predetermined proportion of pixels of the processed image have been set to 1, for example approximately 9%. If less than 9% of the pixels have been set to 1, the validation process indicates that the threshold value was set too high. Consequently the results are considered unsuccessful and the analysis algorithm is repeated for the image with a lower threshold value.

A yet further example of a step which may be carried out in the validation process involves comparing a proportion of pixels set to 1 of an image for one plane of the sample with a proportion of pixels set to 1 of a second, different, plane of the sample. It is unlikely that there will be a significant difference between these two proportions. If the difference when compared with a predetermined difference value, is determined to be statistically significant, the analysis algorithm is then repeated for the images with an appropriately modified value of the threshold to obtain successful results.

Another example of a step which may be carried out in the validation process involves counting a number of isolated pixels set to 1. By isolated it is meant that the pixel is entirely surrounded by pixels set to 0. If this number is higher than a predetermined value, then the validation process indicates that pixels corresponding to background noise of the image have been incorrectly set to 1 and that the analysis algorithm should be repeated for the image with an increased threshold value.

In a further example of a step which may be carried out in the validation process a statistical property, for example an average size or a most probable size, of an identified bacterium of the processed image is compared with a predetermined and expected value for this property. If there is a significant difference between these values, the validation process indicates that the value of the binarisation threshold should be modified and the analysis algorithm repeated. A modified form of this example of the validation process involves comparing the statistical property of an identified bacterium for one image plane of the sample with the statistical property of an identified bacterium for a second, different, image plane of the sample to check for a similarity.

Next, the program determines whether there is another well to process (592). If there is another well, the whole process is started again from process (510), using the first Z-plane (Z=1) of the next well (i=i+1). If there are no other wells to process, the program is finished (593).

FIG. 12 is the flow chart of the subroutine (570) of FIG. 7. This subroutine determines the volume of the biofilm in each colour channel. The subroutine receives, in steps 686, 687 and 688, as input the images of each colour channel from process (561), (562) and (563). Firstly, the subroutine computes three new images, represented using volume elements (voxels), which are simultaneously present in two colour planes (red and green in process (690), red and blue in process (691) and green and blue in process (692)). This is, for example, achieved by taking a logical AND between the images of two colour planes, voxel-to-voxel. Secondly, six different volumes are computed: the volume of the biofilm in each colour plane (red in process (693), green in process (696) and blue in process (698)) and the volume of the overlapping voxels between two colour planes (processes (694), (695), (697)). In order to compute these volumes, it is necessary to know the volume of a voxel. This has been determined in a previous experiment and the value of the voxel volume is stored in the computer memory (689). Subsequently, the sub-routine returns (699) the value of the volumes to the process (571) of the main program.

FIG. 13 is the flow chart of the 3D Object analysis subroutine (581) to (583) that determines the statistical distribution of the geometrical shape parameters of the biofilm clusters and the channels between the clusters. The sub-routine receives as input (700) the Z-stack of image from one of the colour channel from either routine (561), (562) or (563). The analysis of the micro-organism clusters and of the free channels take place in two different flow processes: (720), (730) and (740) for the clusters and (711),(721), (731) and (741) for the channels. For the cluster analysis, firstly, each separate cluster is identified as a separate object in process (720). An object is defined as a group of adjacent voxels that is separated from all the other groups of voxel. This group of voxels occupies a volume in a 3-dimensional space. Process (720) builds a database of objects containing the co-ordinates of all the voxels contained in that object. This database is given as an output to process (584) for further analysis. Secondly, the geometrical properties of each object in the database are determined in process (730). These properties can include, for example, the volume of the object, the length of the long and short axis of the object, the direction cosines of the object, the aspect ratio of the object. Thirdly, the statistical distribution of the shape parameters is computed for the well in process (740). Fourthly, the computed data are stored for further use by process (590). On the other hand, in the free channel analysis flow process, firstly process (711) inverts the image, i.e. the 1 s are converted in Os and vice-versa. Secondly, process (721) identifies the position of the nodes and the connecting links in the network of channels. A node is defined as a point where at least two channels meet and a link is defined as the part of the channel connecting two nodes. The image is therefore converted into a topological database with the co-ordinate of the voxels composing every node and every link. Thirdly, process (731) computes the geometrical properties of all nodes and links, such as number of links connected at a node, length of the link, diameter of a link, curvature of a link, etc. Fourthly, process (741) determines the statistical distribution of the properties of the nodes and the links. Process (741) also computes some global descriptors of the network such as the connectivity, the tortuosity, the fractal dimension, etc. of the network. Fifthly all the results are given as output to process (590).

FIG. 14 is the flow chart of the 3D Object Cross Correlation Analysis sub-process (584). The sub-process receives three inputs. These are the Z-stack for the 3 colour planes of the well. The red stack (801) was stored previously at (572), the green stack (802) was stored previously at (573), the blue stack (803) was stored previously at (574). Firstly, the program computes three new images, corresponding to the voxels that are present simultaneously in, respectively, the red and green images for process (811), the red and the blue images for process (812), the green and the blue images for process (813). These new images are called respectively the Red-Green Overlap image (RGO), the Red-Blue overlap image (RBO) and the Green-Blue overlap image (BGO). This is, for example, achieved by taking a logical AND between the stack of images of two colour planes, voxel-to-voxel. The analysis of the RGO image is carried out in processes (821),(841), (851) and (861) and will be described in more details below. The same analysis is carried out on the RBO image in processes (822), (842), (852) and (862) and the GBO image in the processes (823), (843), (853) and (863). In the case of the RGO image, firstly, each separate cluster of voxels in the stack of images is identified as a separate object in process (821). An object is defined as a group of adjacent voxels that is separated from all the other groups of voxels. This group of voxel occupies a volume in a 3-dimensional space defined by the Z-stack of images. Process (821) builds a database of objects containing the co-ordinates of all the voxels contained in that object. Secondly, process (841) compares the voxel co-ordinates of the objects contained in three databases: the database created by the processes (821), the database of red objects (833) that was created by process (581) and the database of green objects that was created by process (582). If the voxels co-ordinates. of an object in the RGO database are identical to the voxels co-ordinates in the Red object database (833) and the Green object database (832), it is said that there is an overlap between the object in the red and the green stack. The corresponding object in the RGO database is selected. Otherwise the corresponding object in the RGO database is deleted. Thirdly, the geometrical properties of the object from the RGO database are measured in process (851). These properties can include, for example, the volume of the object, the length of the long and short axis of the object, the direction cosines of the object, the aspect ratio of the object, its position with respect to the X,Y and Z axis of the image stack. Fourthly, the statistical distribution of the shape parameters is computed for the well in process (861). Processes identical to (821), (841), (851) and (861) are taking place for the RBO image stack and the GBO image stack.

Finally the RGO database, the RBO database and the GBO database are given as output from the process (590).

### EXAMPLE 1

Visualisation of Bacterial Biofilm by Fluorescence Staining

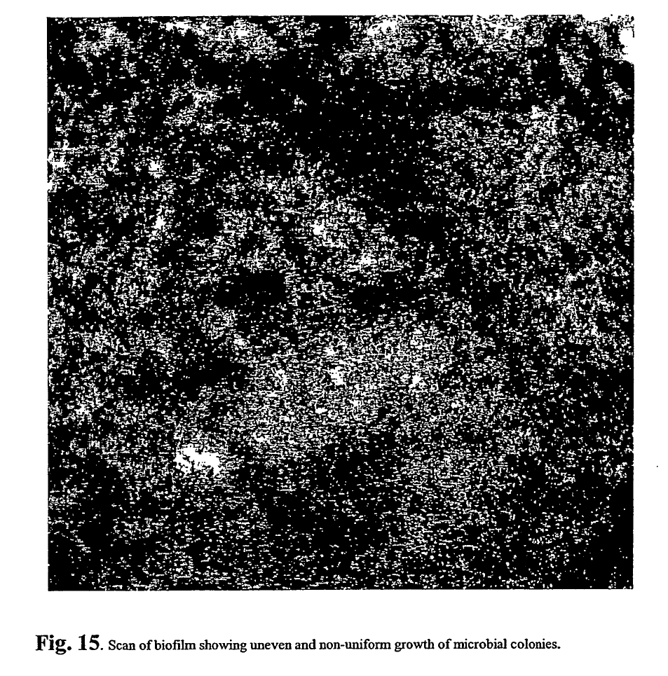

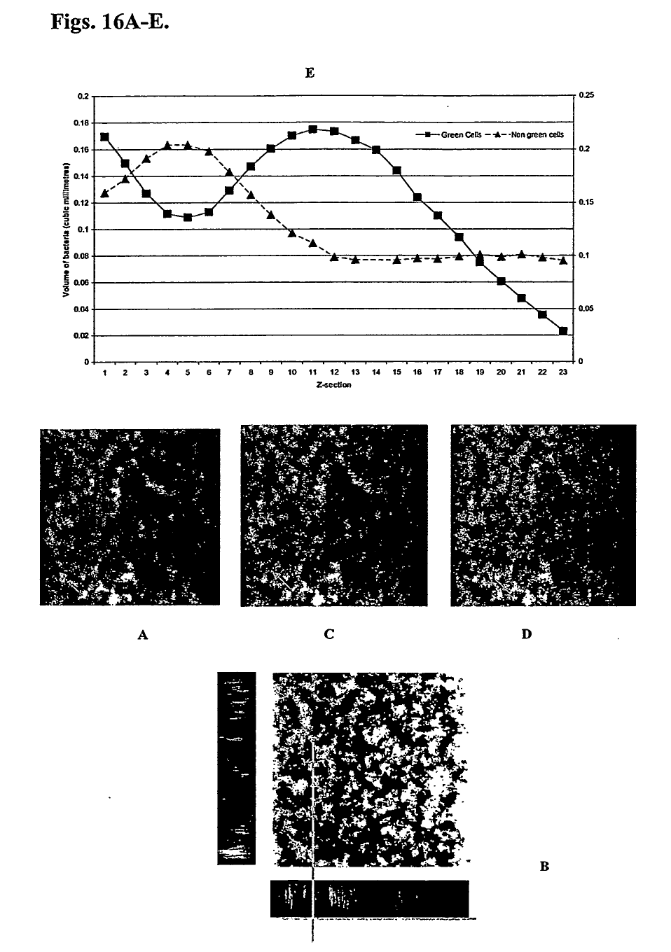

A non-fluorescent strain of Escherichia coli (E. coli, JM109) was allowed to adhere to the wells of a Packard microtitre plate (cf Packard catalogue number 6005182) at 37° C. for 3 hours. Unattached bacteria were removed by washing and the attached cells allowed to grow overnight at 37° C. in standard Luria media (Amersham Biosciences). The bacteria were then visualised by the addition of the fluorescent DNA stain Hoechst 33342 (1 μM; Sigma). On visualisation in the imaging system, the bacteria showed a dense, but not uniform, pattern of staining indicative of growing in patches (see FIG. 15). A scan of the fluorescence intensity into the depth of the biofilm indicated that the film was several micrometers in depth.

### EXAMPLE 2

3D Visualisation of Adhered Population of E. coli Constitutively Expressing GFP

A second experiment was conducted growing E. coli in a microtitre plate as in Example 1 above, except that the E. coli JM 109 constitutively expresses a GFP, having the GFP-F64L-S175G-E222G mutation. After removal of unattached cells by washing, the remaining cells were incubated for approximately 10 hours at 37° C. with no agitation. A well-developed biofilm, that was neither over-grown nor only one-cell thick, was scanned as it was considered representative of a typical sample. Scanning was conducted at 488 mn to visualise the GFP.

The images shown in FIGS. 16A-D cover an area of 0.75×0.75 mm. Depth information was obtained by moving the focal plane of the instrument into the biofilm in 1 μm steps. The three images shown in FIGS. 16A, 16C and 16D represent slices taken and the respective z-position within the biofilm. The image shown in FIG. 16B is a 3-D rendered image, combining 30 images taken. The horizontal and vertical lines representing software ‘cut-lines’ with associated cut profiles to the left and the bottom of the image. The dark areas indicate the absence of biofilm or presence of substantial channels between the microbial clusters which are seen as light areas in the Figure.

FIG. 16E graphically displays the variation of the total intensity of the image as a function of the position of the Z plane.

### EXAMPLE 3

Differential 3D Analysis of Adhered E. coli Cultures

E. coli CL182 possesses a low copy number vector (pGEX-6P-1) that confers ampicillin resistance and expresses the GFP (F64L, S175G, E222G) from the IPTG responsive tac promoter. E. coli XL1-blue is a standard strain that possesses transposon 10, conferring tetracycline resistance. The strains were mixed and grown in the presence of selective antibiotics (tetracycline, ampicillin or chloramphenicol) and the effects were visualised using the IN Cell Analyzer.

E. coli cultures were grown in batch culture at 37° C. for 16 hours under selective pressure. Cells were pelleted by centrifugation, washed and resuspended in cold PBS. OD600 mn was normalised (to 1) for both cultures and solutions were diluted ten fold in cold PBS. Cells (100 μl) were allowed to settle and adhere to the surface of a Packard microtitre plate (Packard #6005182) at 4° C. for 1 hour. Adhered E. coli were washed twice with PBS (100 μl). Luria broth (100 μl) with or without IPTG (0.2 μM), and ampicillin (100 μg/ml), tetracycline (10 μg/ml) and chloramphenicol (34 μg/ml) were added to adhered cells and incubated at 37° C. for 4 hours and 16 hours. Cells were washed twice with PBS and incubated for 10 minutes in Hoechst 33342 (0.1 μM in PBS). Cells were washed in PBS and imaged sequentially using the UV (365 nm) and blue (488 nm) laser lines to excite Hoechst 33342 and GFP respectively. Emission light was captured using the 450BP65 and 565BP50 filters and collected as 12 bit images; 24 slices were acquired per condition with 1 μm spacing between them.

The images were then analysed using the algorithms described in FIGS. 7 to 14. The voxel dimensions were established prior to conducting the experiment using sub-resolution and super-resolution beads. The analysis thus provides data on the amount of biofilm in both colour planes in three dimensions as described previously. In addition, the overlap between green and blue fluorescent biofilm was calculated in 3D.

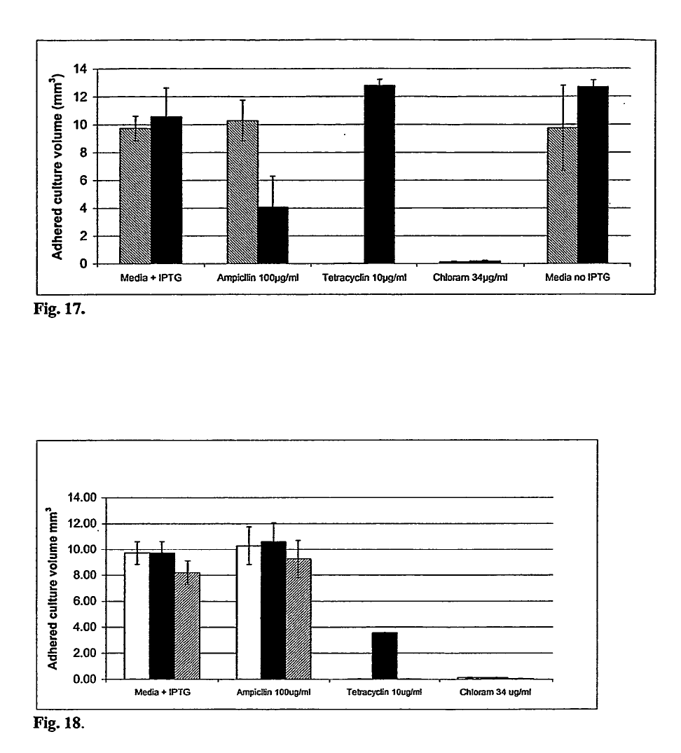

Differential inhibition of growth of XL1-blue and CL182 cells was clearly demonstrated and quantified using ampicillin and tetracycline (FIG. 13). As expected, choramphenicol strongly inhibited the growth of both XL1-blue and CL182. Further, a ˜25% reduction in the expression of GFP from the tac promoter by CL182 was observed in the absence of IPTG. By analysing the voxels overlap positional information on the expression of genes throughout a biofilm is acquired. FIG. 17 shows the mean adhered culture volume (mm³) of the two different strains of E. coli in the presence of antibiotics. The experiment was repeated in four separate wells and the standard deviation between these wells is indicated.

When CL182 cells were incubated in the presence of LB+IPTG the volume of green and blue emission pixels was equal, suggesting that all cells within the adhered biomass volume are expressing GFP (green:blue=9.72:9.71). This was confirmed by further analysis that showed that virtually all pixels that are blue are also green (FIG. 18). Non-GFP expressing, XL1-blue cells nevertheless show intense blue staining in the presence of Hoechst 33342 when grown in LB+IPTG. This allows the discrimination of XL1-blue cells from CL182 cells (GFP expressing).

The above embodiments are to be understood as illustrative examples of the invention. Further embodiments of the invention are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. An automated method for measuring the development of a biofilm, containing one or more fluorescent moieties, on a plurality of surfaces using a confocal imaging system, wherein said confocal imaging system includes
a) means for forming a beam of electromagnetic radiation comprising including one or more wavelengths;
b) means for directing and focusing said beam onto one or more planes of the biofilm;
c) a detection device for detecting electromagnetic radiation emitted from the biofilm; and
d) a scanning device for scanning the biofilm in a plurality of planes with the electromagnetic radiation,
the method comprising the steps of:
i) growing said biofilm on said plurality of surfaces;
ii) detecting the presence of said one or more fluorescent moieties within the biofilm by scanning the biofilm with electromagnetic radiation in a plurality of planes and collecting fluorescent emissions to produce a plurality of images; and
iii) analysing said images by means of a data processing system under the control of computer software to determine the structure of the biofilm.

2. The method of claim 1, wherein:
a) said beam forming means produces an elongated beam of electromagnetic radiation including one or more wavelengths and extending transverse to an optical axis along which the radiation propagates;
b) said directing and focusing means focuses said elongated beam onto a first elongated region in a first plane where the biofilm is located and directs fluorescent radiation emitted from the biofilm onto one or more second elongated regions, wherein each second elongated region is on a different second plane conjugate to the first plane;
c) in at least one of the second conjugate planes, or in a third plane conjugate to at least one of the second conjugate planes, said detection device includes a rectangular array of detection elements on which the electromagnetic radiation emitted from the object is coincident; and
d) the scanning device scans the biofilm by moving the elongated beam relative to the biofilm or by moving the biofilm relative to the elongated beam such that the emitted fluorescent radiation is delivered to the rectangular array of detection elements and is converted by the detection device into a plurality of electrical signals representative of the emitted fluorescent radiation synchronously with said scanning.

3. The method of claim 1, further comprising the step of restoring each image prior to carrying out the image analysis of step iii).

4. The method of claim 1, wherein the beam of electromagnetic radiation produced comprises one or more wavelengths in the range of 350 to 700 nm.

5. The method of claim 1, further comprising the step of measuring the development of the biofilm at a plurality of time points.

6. The method of claim 1, wherein said fluorescent moiety is the product of a gene.

7. The method of claim 6, wherein said gene encodes a fluorescent protein.

8. The method of claim 7, wherein said fluorescent protein is a modified Green Fluorescent Protein (GFP) having at least one mutation selected from the group consisting of Y66H, Y66W, Y66F, S65T, S65A, V68L, Q69K, Q69M, S72A, T203I, E222G, V163A, 1167T, S175G, F99S, M153T, V163A, F64L, Y145F, N149K, T203Y, T203Y, T203H, S202F and L236R.

9. The method of claim 8, wherein said modified GFP has three mutations selected from the group consisting of F64L-V163A-E222G, F64L-S175G-E222G, F64L-S65T-S175G and F64L-S65T-V163.

10. The method of claim 6, wherein the fluorescent moiety is a biosensor capable of monitoring environmental change or enzyme activity within the biofilm.

11. The method of claim 1, wherein the fluorescent moiety is produced by the action of an enzyme on a compound.

12. The method of claim 11, wherein said enzyme is selected from the group consisting of β-galactosidase, nitroreductase, alkaline phosphatase and β-lactamase.

13. The method of claim 1, wherein the method further comprises adding a fluorescent compound to the biofilm before carrying out detection step ii).

14. The method of claim 13, wherein said fluorescent compound is selected from the group consisting of Hoechst 33342, Cy2, Cy3, Cy5, CypHer, coumarin, FITC, DAPI, Alexa 633 DRAQ5, Alexa 488, acridone, quinacridone, fluorescently labelled protein, fluorescently labelled lectin and fluorescently labelled antibody.

15. The method of claim 13, wherein the fluorescent compound is capable of monitoring environmental change within the biofilm.

16. The method of claim 1, wherein said surfaces form a container.

17. The method of claim 16, wherein said container is a microtitre plate.

18. The method of claim 1, wherein the method is capable of determining the size of microbial clusters within the biofilm.

19. The method of claim 1, wherein the method is capable of determining the three-dimensional structure of the biofilm.

20. The method of claim 1, wherein the method is capable of determining the distribution of distances between microbial clusters within the biofilm.

21. The method of claim 1, wherein the method is capable of determining the orientation of microbial clusters within the structure of the biofilm.

22. The method of claim 1, wherein the method is capable of determining the presence of and the size of any channel within the structure of the biofilm.

23. The method of claim 22, wherein the method is capable of determining the connectivity of said channel with any other channel within the structure of the biofilm, thereby defining a channel network.

24. The method of claim 23, wherein the method is capable of determining the fractal dimension of said channel network.

25. The method of claim 1, wherein the biofilm includes optically distinguishable microbial populations.

26. The method of claim 1, wherein the biofilm comprises genetically distinguishable microbial populations.

27. The method of claim 25, wherein the method is capable of determining the spatial distribution of said populations.

28. A method for screening a test agent whose effect upon the development of a biofilm is to be determined, said method comprising the steps of:
i) performing the method of claim 1 in the presence of said test agent; and
ii) comparing the development of the biofilm in the presence of the test agent with a known value for the development of the biofilm in the absence of the test agent,
wherein a difference between the development of the biofilm in the presence of the test agent and said known value in the absence of the test agent is indicative of the effect of the test agent upon the development of the biofilm.

29. The method of claim 28, wherein the known value is stored upon an electronic or optical database.

30. A method for screening a test agent whose effect upon the development of a biofilm is to be determined, the method comprising the steps of:
i) growing said biofilm in the presence and absence of said test agent in a container; and
ii) measuring the development of the biofilm according to the method of claim 1,
wherein a difference in the development the biofilm in the presence and absence of the agent is indicative of the effect the test agent has upon the development of the biofilm.

31. The method of claim 30, wherein said difference in activity between the development of the biofilm in the absence and in the presence of the test agent is normalised, stored optically or electronically and compared with a value of a reference compound.

32. The method of claim 28, wherein the test agent affects gene expression within the biofilm.

33. The method of claim 32, wherein the test agent selectively affects gene expression of specific microbial populations within the biofilm.

34. The method of claim 28, wherein the test agent inhibits biofilm development.

35. The method of claim 28, wherein the test agent promotes the development of the biofilm.

36. The method of claim 28, wherein the test agent is a physical agent selected from the group consisting of electromagnetic radiation, ionising radiation, electric field, sound energy and abrasion.

37. The method of claim 28, wherein the test agent is a fluorescent compound or a fluorescently labelled compound thereby facilitating measurement of its distribution throughout the biofilm.

38. The method of claim 28 wherein the test agent is selected from the group consisting of organic compound, inorganic compound, peptide, polypeptide, protein, carbohydrate, lipid, nucleic acid, polynucleotide and protein nucleic acid.

39. The use of the method of claim I to measure the development of a biofilm

40. A method of analysing the three dimensional structure of an object scanned using a fluorescence imaging system, the system including:
a) a radiation source system for forming a beam of electromagnetic radiation comprising one or more wavelengths;
b) an optical system for directing and focusing said beam onto one or more planes of the object;
c) a detection system for detecting electromagnetic radiation emitted from the object and producing image data; and
d) a scanning system for scanning the object in a plurality of planes with the electromagnetic radiation,
the method comprising scanning the object in a plurality of planes to produce image data including a plurality of images, and processing said image data to determine data relating to a three dimensional structure of the object, the method including an automated image data thresholding step, said thresholding step comprising:
i) analysing intensity values in the image data;
ii) calculating a threshold value for the image data; and
iii) processing the image data using said threshold value to generate thresholded image data.

41. The method of claim 40, wherein said method step i) is arranged to compute statistical properties of the intensity values in the image data.

42. The method of claim 41, wherein during said computing of statistical properties, said method is arranged to compute an intensity histogram, and wherein during said step of calculating a threshold value, said method is arranged to use the intensity histogram to calculate the threshold.

43. The method of claim 41, wherein during said computing of statistical properties, said method is arranged to compute an average intensity and/or an intensity standard deviation.

44. The method of claim 40, wherein said thresholded image data is binarised image data.

45. The method of claim 40, wherein said image data thresholding step further comprises storing at least one validation rule and validating said thresholded image data using said at least one validation rule.

46. The method of claim 45, wherein said at least one validation rule includes a rule relating to a total number of pixels forming an image.

47. The method of claim 45 wherein said at least one validation rule includes a rule relating to a number of pixels forming a predetermined part of an image.

48. The method of claim 45, wherein at least one validation rule includes a rule relating to a number of individually isolated pixels.

49. The method of claim 45, wherein said at least one validation rule includes a rule relating to a proportion of pixels set to above or below the threshold.

50. The method of claim 40, wherein said image data processing has an image data restoration step, said restoration step involving restoring the image data prior to performing said image data thresholding step.

51. The method of claim 40, wherein said object is a biofilm.

52. The method of claim 51, wherein said method is adapted to compare a difference in activity between development of the biofilm in the presence and in the absence of a test agent.

53. Computer software for analysing the three dimensional structure of an object scanned using a fluorescence imaging system, the system including:
a) a radiation source system for forming a beam of electromagnetic radiation comprising one or more wavelengths;
b) an optical system for directing and focusing said beam onto one or more planes of the object;
c) a detection system for detecting electromagnetic radiation emitted from the object and producing image data; and
d) a scanning system for scanning the object in a plurality of planes with the electromagnetic radiation,
wherein said computer software is arranged to perform software steps including controlling the scanning system to scan the object in a plurality of planes to produce image data including a plurality of images, processing said image data to determine data relating to a three dimensional structure of the object, and performing an automated image data thresholding step, said thresholding step comprising:
i) analysing intensity values in the image data;
ii) calculating a threshold value for the image data; and
iii) processing the image data using said threshold value to generate thresholded image data.

54. The computer software of claim 53, wherein said computer software is arranged in step i) to compute statistical properties of the intensity values in the image data.

55. The computer software of claim 54, wherein said computer software is arranged to compute an intensity histogram during said computing of statistical properties, and wherein during said step of calculating a threshold value, said computer software is arranged to use the intensity histogram to calculate the threshold.

56. The computer software of claim 54, wherein said computer software is arranged to compute an average intensity and/or an intensity standard deviation during said computing of statistical properties.

57. The computer software of claim 53 wherein said thresholded image data is binarised image data.

58. The computer software of claim 53, wherein said image data thresholding step further comprises storing at least one validation rule and validating said thresholded image data using said at least one validation rule.

59. The computer software of claim 58, wherein said at least one validation rule includes a rule relating to a total number of pixels forming an image.

60. The computer software of claim 58, wherein said at least one validation rule includes a rule relating to a number of pixels forming a predetermined part of an image.

61. The computer software of claim 58, wherein said at least one validation rule includes a rule relating to a number of individually isolated pixels.

62. The computer software of claim 58, wherein said at least one validation rule includes a rule relating to a proportion of pixels set to above or below the threshold.

63. The computer software of claim 53, wherein said image data processing step has an image data restoration step, said restoration step involving restoring the image data prior to performing said image data thresholding step.

64. The computer software of claim 53, wherein said object is a biofilm.

65. The computer software of claim 64, wherein said computer software is adapted to compare a difference in activity between development of the biofilm in the presence and in the absence of a test agent.

66. A data carrier storing the computer software of claim 53.
